SEQUENCE LISTING (3 sequences)

SEQ ID NO: 1 - DNA, 11 bp, Artificial
  Synthetic DNA fragment
gttagggttag

SEQ ID NO: 2 - DNA, 16 bp, Artificial
  Synthetic DNA Fragment
gttagggttagggtta

SEQ ID NO: 3 - DNA, 16 bp, Artificial
  Synthetic DNA Fragment
ggttggttggttggtt

(19) United States
(12) Patent Application Publication
(10) Pub. No.: US 2010/0093716 A1
(43) Pub. Date: Apr. 15, 2010

(54) THERAPEUTIC METHODS USING WRN BINDING MOLECULES

(75) Inventors: Barbara A. Gilchrest, Boston, MA (US); Mark S. Eller, Boston, MA (US); Angela N. Koehler, Cambridge, MA (US); Olivia M. McPherson, Cambridge, MA (US); Christopher S. Neumann, Cambridge, MA (US); Timothy A. Lewis, Marlborough, MA (US)

Correspondence Address: HOWREY LLP - East, C/O IP DOCKETING DEPARTMENT, 2941 FAIRVIEW PARK DR, SUITE 200, FALLS CHURCH, VA, 22042-2924, US

(73) Assignee: TRUSTEES OF BOSTON UNIVERSITY AND PRESIDENT AND FELLOWS OF HARVARD COLLEGE, Boston, MA (US)

(21) Appl. No.: 12/438,793

(22) PCT Filed: Aug. 29, 2007
(86) PCT No.: PCT/US07/77147

§ 371 Date: Dec. 21, 2009

Related U.S. Application Data
(60) Provisional application No. 60/823,876, filed on Aug. 29, 2006.

(51) Int. Cl. A61K 31/5386; C07D 498/20; A61P 35/04

(52) U.S. Cl. 514/229.5; 544/70

(57) ABSTRACT

The present invention provides, inter alia, compositions and methods for treating various diseases and disorders in a mammal by administering to a mammal in need an effective amount of a composition comprising a non-DNA small molecule that binds WRN, such as members of the spirooxindole (SPOX) class.

### CROSS-REFERENCE TO RELATED APPLICATIONS

The present application claims the benefit of U.S. Provisional Patent Application No, 60/823,876, filed Aug. 29, 2006, the contents of which are incorporated herein by reference.

### FIELD OF THE INVENTION

The present invention relates to compositions and methods for the regulation of signaling pathways. More specifically, the present invention relates to, inter alia, compositions and methods for the regulation of telomere-initiated senescence, apoptosis, tanning and other DNA damage responses.

### BACKGROUND OF THE INVENTION

The frequency of cancer in humans has increased in the developed world as the population has aged. For some types of cancers and stages of disease at diagnosis, morbidity and mortality rates have not improved significantly in recent years in spite of extensive research. During the progression of cancer, tumor cells become more and more independent of negative regulatory controls, including resistance to senescence and apoptosis, important aspects of how the interaction of normal cells with their tissue-specific environment is regulated.

In germline cells and most cancer cells, immortality is associated with maintenance of telomere length by telomerase, an enzyme complex that adds TTAGGG repeats to the 3′ terminus of the chromosome ends. Telomeres, tandem repeats of TTAGGG, end in a loop structure with a 3′ single-stranded overhang of approximately 150-300 bases tucked within the proximal telomere duplex DNA and stabilized by telomeric repeat binding factors (TRFs), particularly TRF2. Ectopic expression of a dominant-negative form of TRF2 (TRF2^{DN}) disrupts telomere loop structure, exposes the 3′ overhang and causes DNA damage responses. Depending on the cell type, cells then undergo senescence as in the case of primary fibroblasts, fibrosarcoma cells, and several other malignant cell types or apoptosis as in the case of lymphocytes.

Evidence implicates progressive telomere shortening (caused by an inability to replicate the 3′ ends of chromosomes) or some other form of telomere dysfunction in senescence. Ectopic expression of the telomerase reverse transcriptase catalytic subunit (TERT), which enzymatically maintains or builds telomere length, can bypass senescence with subsequent immortalization of some human cell types, strongly suggesting a telomere-dependent mechanism of replicative senescence. Moreover, malignant cells commonly express TERT and/or contain mutations that allow the cell to bypass the senescent response and to proliferate indefinitely despite often having shorter telomeres than normal senescent cells. However, some tumor cells undergo senescence in response to various anticancer agents, indicating that acquisition of immortality does not necessarily imply a loss of this basic cellular response to DNA damage.

Senescence and apoptosis in human cells are largely dependent on the p53 pathway. The tumor suppressor p53 plays a key role in cellular stress response mechanisms by converting a variety of different stimuli, for example, DNA damage, deregulation of transcription or replication, oncogene transformation, and deregulation of microtubules caused by some chemotherapeutic drugs, into cell growth arrest or apoptosis. When activated, p53 causes cell growth arrest or a programmed, suicidal cell death (apoptosis), which in turn acts as an important control mechanism for genomic stability. In particular, p53 controls genomic stability by eliminating genetically damaged cells from the cell population, and thus one of its major functions is to prevent tumor formation.

An intact tumor suppressor pRb pathway also contributes to preventing tumorigenesis. In pRb^{-/-} tumor cells that do not contain wild-type p53, introduction of pRb induces senescence. Although cervical cancer cells frequently retain wild-type p53 and pRb genes, the HPV E6 and E7 proteins interfere with the p53 and pRb pathways, respectively. Ectopic expression of viral E2 protein represses HPV E6 and E7 gene transcription and induces a rapid and prominent senescent response in cervical carcinoma cell lines, again affirming the important roles of p53 and pRb in cancer cell senescence.

Suppressing only the p53 or the pRb pathway is not sufficient for fibroblasts to bypass replicative senescence. Indeed, human fibroblasts either transfected with SV 40 T antigen or transduced with combinations of adenovirus E1A+E1B or HPV E6+E7, suppressing both the p53 and pRb pathways, have an extended life span and escape replicative senescence.

Double strand breaks in DNA are extremely cytotoxic to mammalian cells. The highly conserved Mre11-Rad50-NBS(p95) (MRN) complex is involved in the repair of double strand breaks in eukaryotes. The MRN complex adheres to sites of double strand breaks immediately following their formation. The MRN complex also migrates to telomeres during the S-phase of the cell cycle associates with telomeric repeat binding factors (TRF).

The MRN complex consists of Mre11, Rad50 and NBS (p95). Mre11, as part of the Mre11/p95/Rad50 complex, associates with the telomere during S phase of the cell cycle. Mre11 is an exonuclease with preference for the 3′ end of a DNA strand. The activity of Mre11 is believed to be dependent on interaction with Rad50, which is an ATPase. Nbs1 is believed to be involved in the nuclear localization of the MRN complex, as well as its assembly at the site of a double strand break.

A protein mutated in Werner's Syndrome, the WRN protein, is known to interact with the MRN complex (Cheng et al., 2004, Vol. 2004). Werner's Syndrome is an autosomal recessive disorder that is characterized by premature aging, increased malignancies and genomic instability. WRN is a nuclear protein that contains both helicase and 3′ to 5′ exonuclease domains (Oshima, J., 2002, Bioessays 22, 894-901). To date, all mutations identified in Werner's Syndrome are WRN truncations that eliminate the nuclear localization signal from the COOH end of the protein (Oshima, J., 2002). Therefore, it is believed that WRN mutations in Werner's Syndrome generate a functional null phenotype by preventing the protein from reaching its site of action in the nucleus. Cells from Werner's Syndrome patients show increased levels of deletions and translocations, both baseline and after DNA damage, suggesting that the WRN protein participates in DNA repair, replication and recombination (Opresko et al., 2003, Carcinogenesis 24, 791-802). Werner's Syndrome cells also senesce prematurely compared to age-matched controls (Martin et al., 1970, Lab Invest 23, 86-92) and also demonstrate accelerated telomere shortening (Schulz et al., 1996, Hum Genet. 97, 750-4).

In addition to interacting with the MRN complex, WRN is known to interact with other proteins that participate in DNA damage responses and DNA repair/replication: DNA-PK/Ku (Karmakar et al., 2002, Nucleic Acids Res 30, 3583-91), p53 (Brosh et al., 2001, J Biol Chem 276, 35093-102), and the helicase mutated in the premature aging syndrome, Bloom's Syndrome, BLM (von Kobbe et al., 2002, J Biol Chem 277, 22035-44). Furthermore, WRN interacts with telomere repeat-binding factor 2, TRF2, and this interaction alters the specificity of the WRN exonuclease activity to facilitate 3′ to 5′ digestion of the telomeric DNA (Machwe et al., 2004, Oncogene 23, 149-56; Opresko et al., 2002, J Biol Chem 277, 41110-9). Together, these data demonstrate a critical role for WRN in DNA metabolism and telomere maintenance. However the precise role of WRN in these pathways is not understood.

Cancers are typically treated with highly toxic therapies, such as chemotherapy and radiation therapy, that comparably damage all proliferative cells whether normal or malignant. Side effects of such treatments include severe damage to the lymphoid system, hematopoietic system and intestinal epithelia, as well as hair loss. Previously, we disclosed a method of screening for modulators of WRN that may be used to induce growth arrest, apoptosis, and proliferative senescence as shown in PCT/US2005/017553, which is incorporated by reference in its entirety. We also previously discovered that telomere homolog oligouncelotides (T-oligos) mimic disruption of the telomere loop structure and thus, when provided to cells in culture or locally or systematically to intact animals, activate innate cancer-avoidance mechanisms within cells. Activating these DNA damage-like responses in malignant cells causes them to undergo apoptosis or senescence, but causes only transient growth arrest and “adaptive differentiation” of normal cells. T-oligos thus appear to provide a novel and very selective approach to preventing and treating a wide variety of cancers, as well as a means of addressing other unmet medical and cosmetic needs through the stimulated protective “differentiation” responses (e.g. sunless tanning, enhanced DNA repair capacity, and transient immunosuppression for treatment of psoriasis and eczema).

### SUMMARY OF THE INVENTION

The present invention is directed to a method of treating a hyperproliferative disorder in a mammal, comprising administering to the mammal a composition comprising an effective amount of a spirooxindole (SPOX). The SPOX compound may be SPOX-1, SPOX-2, or any other member of the SPOX class capable of binding or interacting with WRN. Also provided is a composition comprising an effective amount of a SPOX compound for use in treating a hyperproliferative disorder in a mammal. Preferably, the mammal is human.

The present invention also provides novel SPOX compounds. More particularly the present invention provides compounds having the general formula:

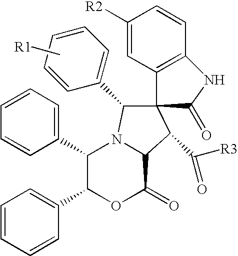

or a pharmaceutically acceptable salt thereof, wherein: R1 is a functional group which may be in the ortho, meta or preferably para position including, but not limited to: hydroxy; lower alkyl; lower hydroxyalkyl such as hydroxymethyl or hydroxyethyl; lower alkoxy such as methoxy, ethoxy, propoxy; and a hydroxy-substituted lower alkoxy such as 2-hydroxyethoxy; R2 is a functional group including, but not limited to: hydrogen; lower alkyl; lower alkyl halide such as iodomethane; halogen such as chlorine, bromine or, preferably, iodine; lower alkenyl; and preferably lower alkynyl, more preferably substituted lower alkynyl, for example, an alkynyl including one or more functional groups such as aryl, arylheterocyclic ring; hydroxy, amino, substituted amino, such as alkylamino, arylamino and carboxamidoamino, ester, carboxamido, alkyl, cycloalkyl, alkenyl, and cycloalkenyl; and R3 is a functional group including, but not limited to: hydroxy, lower alkyl; lower alkynyl; lower alkenyl; amino; substituted amino such as an alkenylamino, preferably allylamino; heterocyclic ring; arylheterocylic ring; lower alkoxy; and lower alkenoxy, preferably allyloxy. The novel SPOX compounds may be used in accordance with the present invention.

In another embodiment, the present invention provides pharmaceutical compositions comprising a SPOX compound of the general formula and a pharmaceutically acceptable carrier.

In another embodiment, the present invention relates to a method of inhibiting growth of cancer cells in a human comprising administering to the human a composition comprising an effective amount of a SPOX compound. The method of this embodiment may result in S-phase arrest in the treated cells followed by apoptosis and/or senescence that is independent of the presence or activity of telomerase and does not require p3 in the cancer cells. Illustrative cancer cells that may be treated with the SPOX compound according to the present invention include melanoma cells, breast cancer cells, lymphoma cells, osteosarcoma cells, leukemia cells, squamous carcinoma cells, cervical cancer cells, ovarian cancer cells, pancreatic cancer cells, lung cancer cells and fibrosarcoma cells. In another embodiment, the SPOX compound may be linked to a targeting molecule that preferentially delivers the compound to cells of interest. Also provided is a composition comprising an effective amount of a SPOX compound for use in inhibiting the growth of cancer cells in a human.

The present invention also relates to a method of promoting differentiation of malignant cells in mammals, comprising administering to the mammal a composition comprising an effective amount of a SPOX compound. The SPOX compound may be combined with growth factors to enhance the differentiation of stem cell cultures in tissue engineering applications. Also provided is a composition comprising an effective amount of a SPOX compound for use in promoting differentiation of malignant cells in mammals. Preferably, the mammal is human.

The present invention also relates to a method of inducing apoptosis in cancer cells in a human, comprising administering to the human a composition comprising an effective amount of a SPOX compound. Illustratively, the cancer cells treated by the method may be melanoma cells or any other cancer cells, for example, those described above. Also provided is a composition comprising an effective amount of a SPOX compound for use in inducing apoptosis in cancer cells in a human.

The present invention also relates to a method of inducing senescence in cancer cells in a human, comprising administering to the human a composition comprising an effective amount of a SPOX compound. Illustratively, the cancer cells treated by the method may be melanoma cells or any other cancer cells, for example those described above. Also provided is a composition comprising an effective amount of a SPOX compound for use in inducing senescence in cancer cells in a human.

The present invention also relates to a method of treating and/or preventing a skin disorder in mammals, comprising administering to the mammal a composition comprising an effective amount of a SPOX compound. The skin disorder may include, but is not limited to: spongiosis, blistering, dyskeratosis (sunburn); melanoma; actinic keratosis; Bowen's disease; vitiligo; squamous cell carcinoma; or basal cell carcinoma. Also provided is a composition comprising an effective amount of a SPOX compound for use in treating a skin disorder in mammals.

The present invention also relates to a method of sunless tanning in a human, comprising administering to the human a composition comprising an effective amount of a SPOX compound. The invention is also directed to cosmetic compositions comprising SPOX compounds for use in reducing photoaging, including tanning, and reducing oxidative damage to skin.

The invention is also directed to methods of identifying therapeutic agents, comprising contacting a candidate agent with a WRN protein or one or more proteins of the MRN complex and measuring binding of the candidate agent to the WRN protein or one or more proteins of the MRN complex whereby a therapeutic agent is identified by its ability to bind to WRN or one or more proteins of the MRN complex.

The invention is also directed to a pharmaceutical composition comprising a compound that binds to the WRN protein or one or more proteins of the MRN complex and a pharmaceutically acceptable carrier. The composition may be useful in accordance with any of the preceding methods.

These and other embodiments of the present invention are described in further detail herein below.

### BRIEF DESCRIPTION OF THE DRAWINGS

FIGS. 1A-1Z shows chemical structures of SPOX compounds known to bind WRN.

FIGS. 2A-2E show the chemical structure of SPOX-1, SPOX-2, SPOX-343, SPOX-338 and SPOX-337.

FIGS. 3 and 4 show the effect of SPOX-1 compared to T-oligo and diluent alone on γH2AX formation in human fibroblasts by immunofluorescent microscopy.

FIG. 5 shows the effect of SPOX-1 and SPOX-2 compared to T-oligo on the growth of newborn fibroblasts.

FIG. 6 shows FACS analysis of propidium iodide stained MM-AN human melanoma cells treated with diluent alone, T-oligo, SPOX-1 and SPOX-2.

FIG. 7 shows the effect of SPOX-1 and SPOX-2 on the growth of MM-AN human melanoma cells.

FIG. 8 shows the effect of SPOX-2 compared to T-oligo and diluent alone on phosphorylation of ATM at serine 1981 in MM-AN human melanoma cells.

FIG. 9 shows the effect of SPOX-1 and SPOX-2 on the growth of MCF-7 cells.

FIG. 10 shows the effect of SPOX-1 and SPOX-2 on the expression of γH2AX by western blot analysis.

FIG. 11 shows the effect of SPOX-1 and SPOX-2 on the expression of cleaved and uncleaned poly(ADP-ribose) polymerase (PARP) by western blot analysis.

FIG. 12 shows FACS analysis of propidium iodide stained WRN+ and WRN− U20S cells treated with positive and negative controls and SPOX-1.

FIG. 13 shows the effect of SPOX-1 and SPOX-2 on melanogenesis in human skin explants.

FIG. 14 is a graphical depiction of the data presented in FIG. 12.

FIG. 15 shows the effect of SPOX-1 and SPOX-2 compared to T-oligo on survivin expression in H460 human lung cancer cells.

FIG. 16 shows the effect of SPOX-1 and SPOX-2 on the growth of H460 human lung cancer cells.

FIG. 17 shows the effect of SPOX compounds SPOX-337, SPOX-338 and SPOX-343 on the growth of MCF-7 cells.

FIG. 18 shows FACS analysis of propidium iodide stained MM-AN human melanoma cells treated with diluent alone, T-oligo, SPOX compounds SPOX-337 and SPOX-338 and SPOX-343.

### DETAILED DESCRIPTION OF THE INVENTION

While the present invention has multiple embodiments, the description of the embodiments set out below is made with the understanding that the present disclosure is to be considered as an exemplification of the invention, and is not intended to limit the invention to the specific embodiments illustrated. Headings are provided for convenience only and are not to be construed to limit the invention in any way. Embodiments illustrated under any heading may be combined with embodiments illustrated under any other heading.

The use of numerical values in the various ranges specified in this application, unless expressly indicated otherwise, are stated as approximations as though the minimum and maximum values within the stated ranges were both preceded by the word “about.” In this manner, slight variations above and below the stated ranges can be used to achieve substantially the same results as values within the ranges. Also, the disclosure of a range is intended as if it were the disclosure of a continuous range including every value between the minimum and maximum values recited as well as any ranges that can be formable by such values.

It is also to be understood that any ranges, ratios and ranges of ratios that can be formed by any of the values or data present herein represent further embodiments of the present invention. This includes ranges that can be formed that do or do not include a finite upper and/or lower boundary.

### SPOX Compounds

Screening for Non-DNA substitutes for T-oligos was performed by testing a library of 6000 SPOX compounds for their ability to bind WRN. Screening for compounds that bind to WRN was conducted using small molecule microarray assays described, for example, in Koehler et al., J. American Chem. Soc., 2003, 125, 8420-8421, and Bradner et al., 2006, Chemistry and Biology, 13, 493-504. Briefly, the library of candidate compounds was printed onto glass slides according to methods described in Koehler, et al., and Bradner, et al. Purified WRN protein was exposed to the library on the slides. After an appropriate incubation period, the slides were washed to remove any unbound WRN. Binding of WRN to the members of the library was detected using antibodies that bind to WRN. Candidate therapeutics were identified by their ability to bind to WRN.

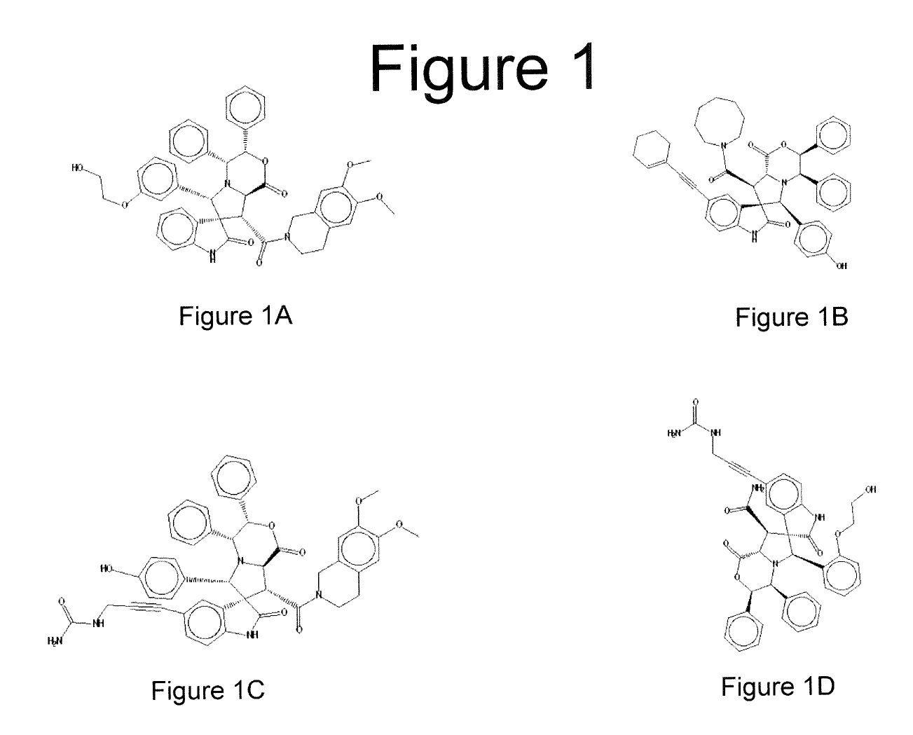

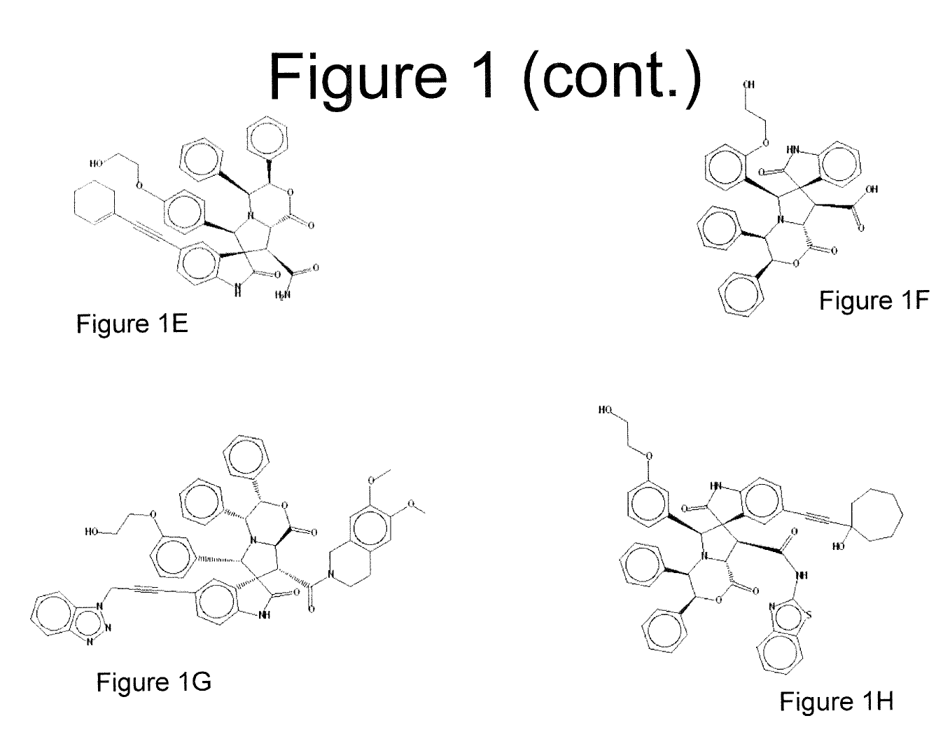

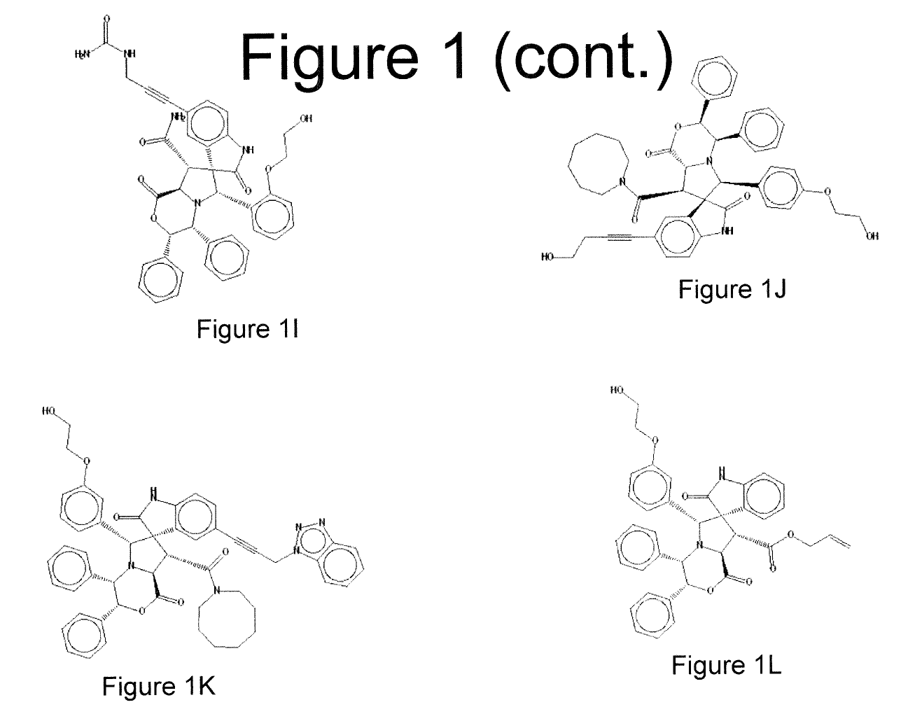

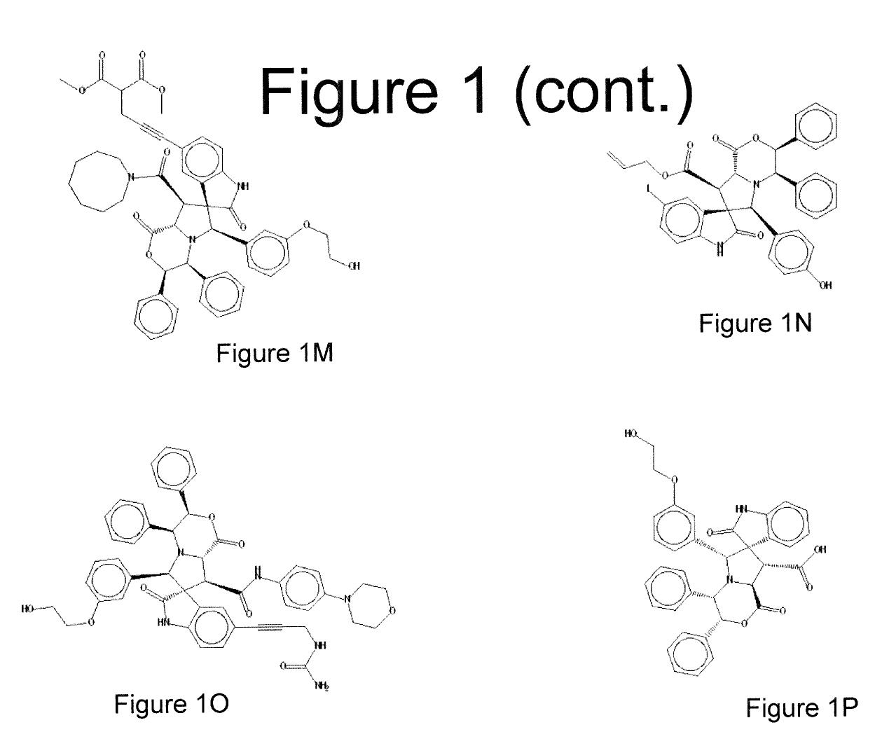

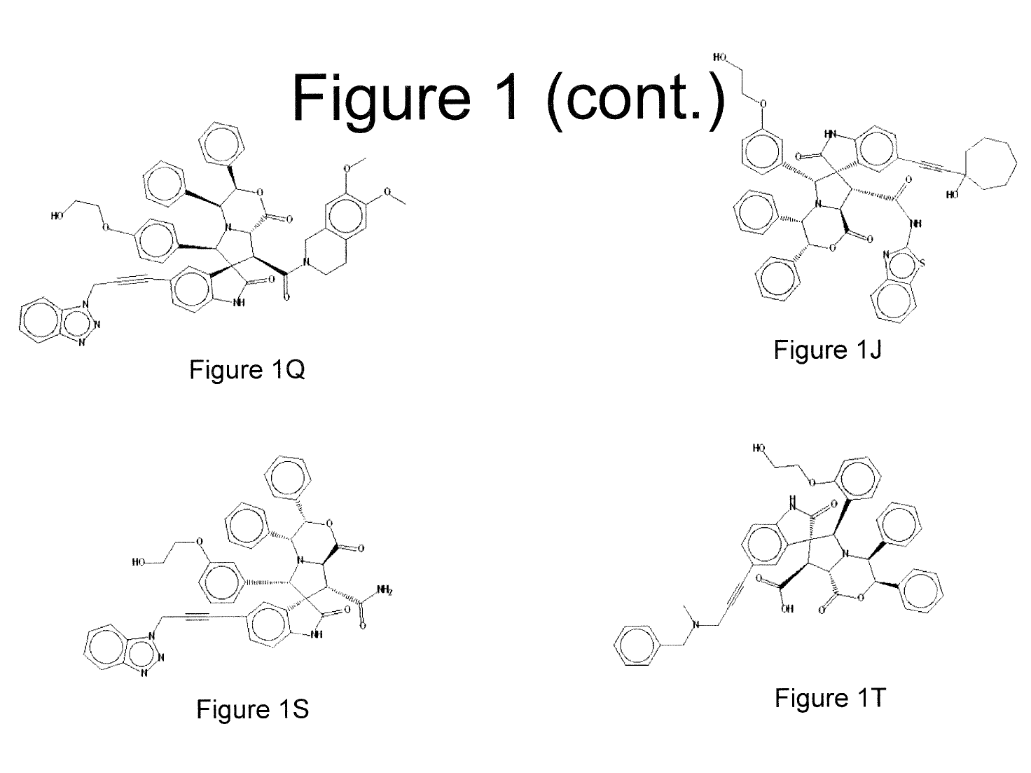

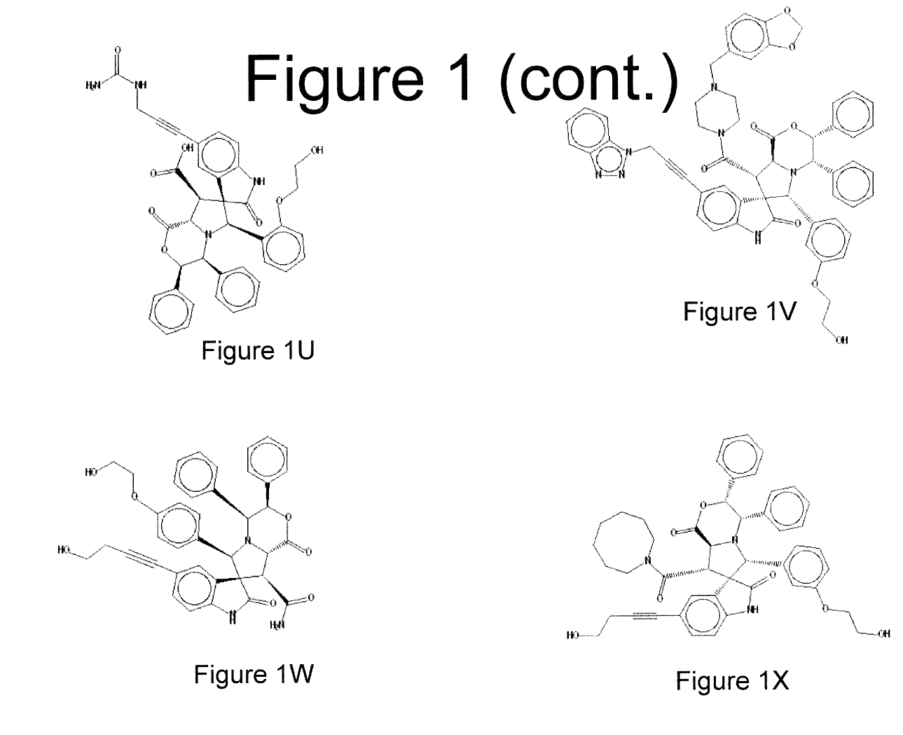

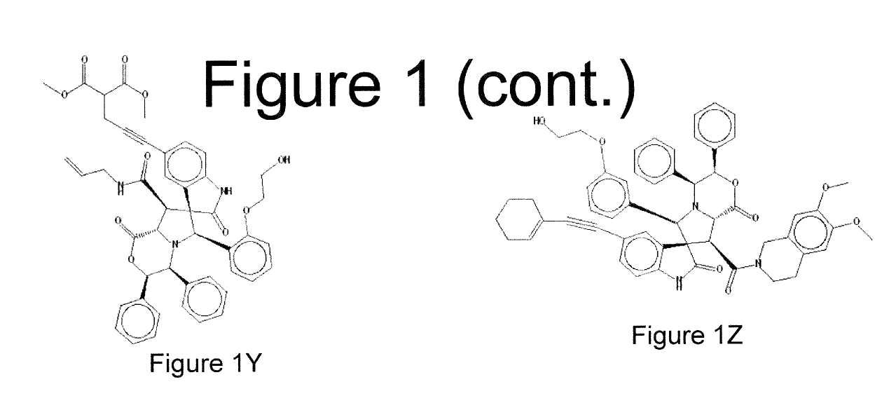

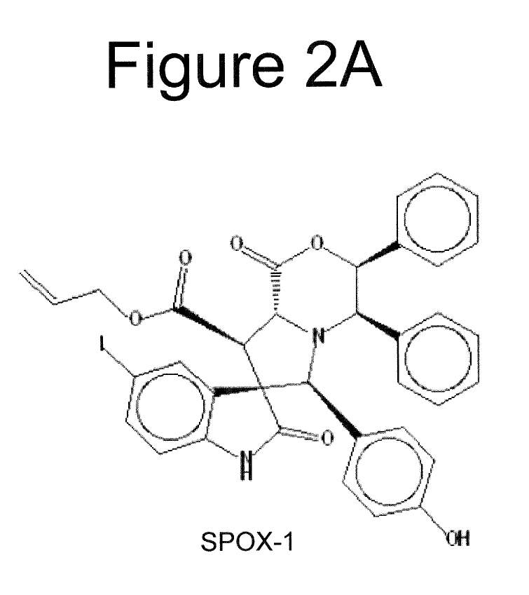

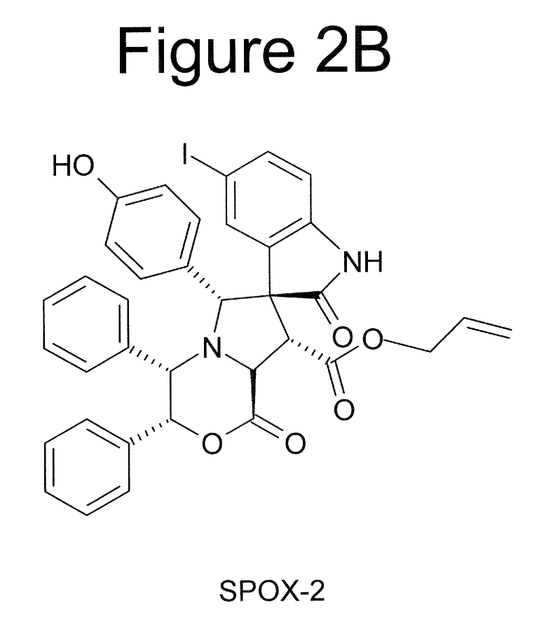

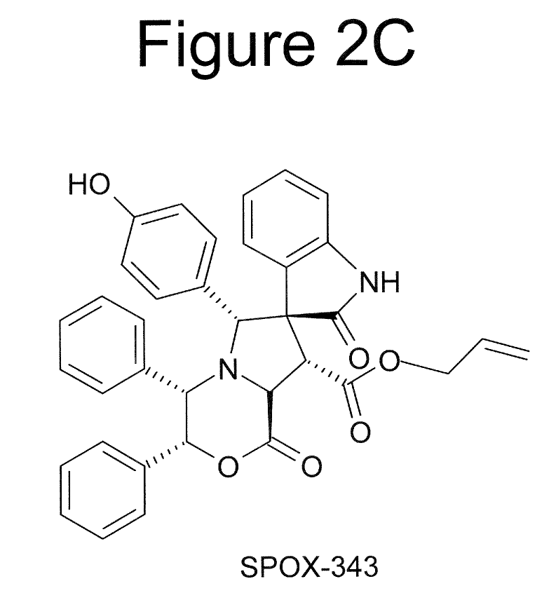

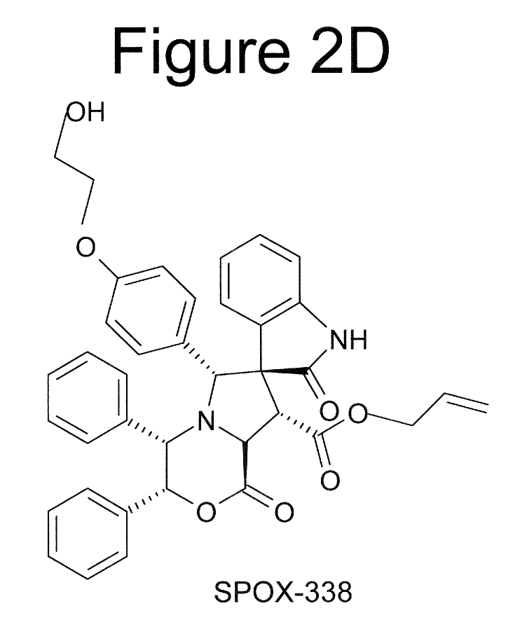

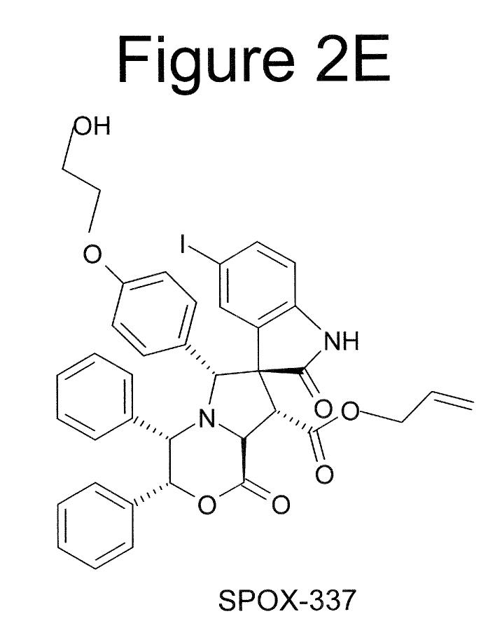

Twenty-six SPOX compounds were found to uniquely and repeatedly bind WRN in these arrays. These compounds were previously not known to bind WRN or to initiate therapeutic responses such as growth arrest and apoptosis of malignant cells. FIG. 1 shows the chemical structures for a number of SPOX compounds identified by the small molecule microarray assay. FIG. 2A shows the compound termed “SPOX-1.”

The library of 6,000 SPOX compounds was chosen based on an initial screening of 35,000 compounds from many different sources and comprising several different scaffolds (core structures) in which it was surprisingly determined that a subset of the 35,000 compounds, having in common a spirooxindole core, bound to WRN.

Studies comparing the efficacy of SPOX-1 with that of certain T-oligos in inducing apoptosis, growth arrest, and pigment expression were performed. T-oligos capable of modulating WRN activity were disclosed in co-pending U.S. patent application Ser. No. 10/122,630, filed Apr. 12, 2002, which is incorporated herein by reference.

SPOX-1 served as the starting point for chemical modifications which led to the identification of several additional SPOX compounds capable of growth arrest and/or apoptosis of malignant cells. FIGS. 2B-E show examples of these compounds. FIG. 2B shows SPOX-2, an enantiomer of SPOX-1. SPOX-2 served as the basis for further modifications resulting in, inter alia, SPOX-337. SPOX-338 and SPOX-343 depicted in FIGS. 2C-E. Neither SPOX-1 nor SPOX-2 exhibits ideal “drugability” properties, i.e. good pharmaceutical properties related to administration, distribution, metabolism and excretion. For example SPOX-1 has a molecular weight of 712, whereas drugs predominantly have molecular weights between 200 and 500. SPOX-1 has a clog (calculated log) P of 6.88 whereas drugs predominantly exhibit clog P values between 2 and 5. P is the ratio of the solubility of a compound in water relative to its solubility in 1-octanol. Hence, clog P is a measure of lipophilicity. Generally compounds which are too water soluble (low clog P) will not enter cell membranes and compounds which are too lipophilic (high clog P) will not leave cell membranes. Thus, SPOX-1 and SPOX-2 may suffer due to their pharmacological properties, although high molecular weight and lipophilicity may be advantageous for topical applications.

Modifications to SPOX-2 were designed to retain or enhance the biological activity of SPOX-1 and SPOX-2 and to achieve desirable properties related to “drugability.” It is to be understood that clog P values between 2 and 5 and molecular weight between 200 and 500 are examples of properties which improve a candidate compound's “drugability.” Compounds SPOX-337, SPOX-338 and SPOX-343 have decreased molecular weight and lower clog P values than SPOX-1 and SPOX-2 and retain the capability of arresting the growth of malignant cells.

The methods described above may be used to identify other classes or types of molecules that interact with WRN according to the present invention which may have therapeutic effects similar to those seen with T-oligos and SPOX, such as the ability to inhibit growth of tumor cells, to induce apoptosis in tumor cells and to induce melanogenesis.

The invention is also directed to methods for identifying therapeutic compounds by virtue of their ability to bind to the WRN protein the method comprising contacting the WRN with a candidate therapeutic compound, and measuring binding of WRN to the compounds or vice versa. A therapeutic agent is identified by its ability to bind WRN. It should be noted that therapeutic agents that bind to WRN may also exert their therapeutic effects through other physiological pathway while nevertheless having the ability to bind to WRN.

The MRN complex is known to interact with WRN. Similar screening methods for identifying a therapeutic may be undertaken by measuring the binding or interaction of candidate therapeutic molecules with one or more of the proteins of the MRN complex including MRE11, Rad50, NBS(p95) according to methods, such as the small molecule microarray assays, described herein.

The invention is also directed to novel SPOX compounds, that may be used in accordance with the present invention, having the general formula:

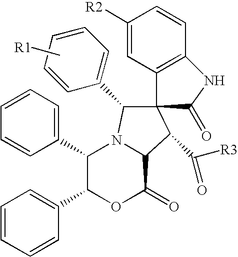

or a pharmaceutically acceptable salt thereof, wherein: R1 is a functional group which may be in the ortho, meta or preferably para position including, but not limited to: hydroxy; lower alkyl; lower hydroxyalkyl such as hydroxymethyl or hydroxyethyl; lower alkoxy such as methoxy, ethoxy, propoxy; and a hydroxy-substituted lower alkoxy such as 2-hydroxyethoxy; R2 is a functional group including, but not limited to: hydrogen; lower alkyl; lower alkyl halide such as iodomethane; halogen such as chlorine, bromine or, preferably, iodine; lower alkenyl; and preferably lower alkynyl, more preferably substituted lower alkynyl, for example, an alkynyl including one or more functional groups such as aryl, arylheterocyclic ring; hydroxy, amino, substituted amino, such as alkylamino, arylamino and carboxamidoamino, ester, carboxamido, alkyl, cycloalkyl, alkenyl, and cycloalkenyl; and R3 is a functional group including, but not limited to: hydroxy, lower alkyl; lower alkynyl; lower alkenyl; amino; substituted amino such as an alkenylamino, preferably allylamine; heterocyclic ring; arylheterocylic ring; lower alkoxy; and lower alkenoxy, preferably allyloxy.

The term “lower alkyl” refers to saturated monovalent aliphatic radicals with 1 to 6 carbons having straight, cyclic or branched moieties. Examples of alkyl radicals useful in the invention include methyl, ethyl, n-propyl, isopropyl, n-butyl, isobutyl, pentyl, hexyl and the like.

The term “lower alkenyl” refers to unsaturated aliphatic moieties with 1 to 6 carbons having at least one carbon-carbon double bond and including E and Z isomers of said alkenyl moiety. Examples of alkenyl radicals include ethenyl, propenyl, butenyl and the like.

The term “lower alkynyl” refers to unsaturated aliphatic moieties with 1 to 6 carbons having at least one carbon-carbon triple bond and includes straight and branched chain alkynyl groups. Examples of alkynyl radicals include ethynyl, propynyl, butynyl and the like.

The term “lower alkoxy” refers to the —OR group where R is an alkyl with 1 to 6 carbons and where R may be a substituted lower alkyl (e.g. hydroxyethoxy).

The term “lower alkenoxy” refers to the —OR group where R is an alkenyl with 1 to 6 carbons and where R may be a substituted lower alkenyl.

The term “halogen” is used herein to refer to fluorine, bromine, chlorine and iodine atoms.

The term “hydroxy” is used herein to refer to the group —OH.

The term “aryl” is used herein to refer to a mono- or bicyclic carbocyclic ring system having one or two aromatic rings, including but not limited to, phenyl, naphthyl, tetrahydronaphthyl, indanyl, indenyl, and the like. The aryl group may be substituted with one two or three substituents independently selected from lower alkyl, haloalkyl, alkoxy, preferably methoxy, halo, hydroxy, nitro, amino and the like.

The term “heterocylic ring” is used herein to refer to any 3-, 4-, 5-, 6-, 7-, 8-, 9- or 10-membered nonaromatic saturated or unsaturated ring containing at least one oxygen, sulfur or preferably nitrogen atom which is bonded to an atom which is not part of the heterocyclic ring. The heterocyclic ring may also be substituted with other groups such as 3,4-methylenedioxyphenyl-2-methyl, benzyl, phenoxy, methoxy and the like. As examples of the heterocyclic ring there can be mentioned octahydroazocinyl, piperazinyl, including 4-(3,4-methylenedioxyphenyl-2-methyl)piperazinyl and the like.

The term “arylheterocyclic ring” is used herein to refer to a bi- or tricyclic ring comprised of an aryl ring as previously defined appended via two adjacent carbons of the aryl group to a heterocylic ring as previously defined. The arylheterocyclic ring may also be substituted with other groups such as 3,4-methylenedioxyphenyl-2-methyl, benzyl, phenoxy, methoxy and the like. As examples of the arylheterocylic ring there can be mentioned isoquinolinyl, including 6,7-dimethoxyisoquinolinyl, and the like.

Within the general formula, certain embodiments are preferred, namely those in which:

R1 is at the ortho, meta or para position and is —OH or —O—CH₂—CH₂—OH;

R2 is selected from the group consisting of:

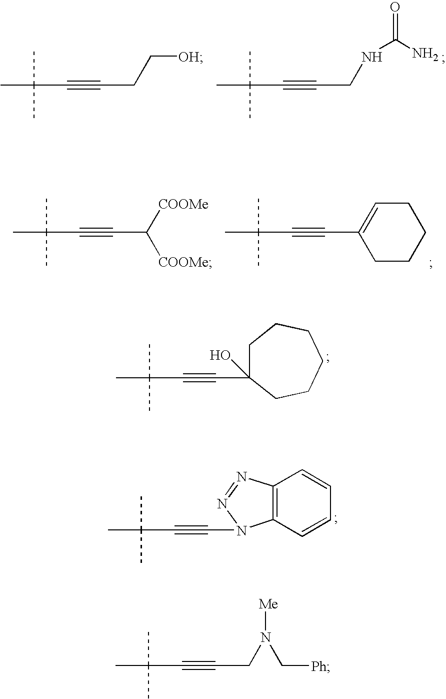

### H; and I; and

R3 is selected from the group consisting of: —OH; —NH₂; —O—CH₂—CH═CH₂; —NH—CH₂—CH═CH₂;

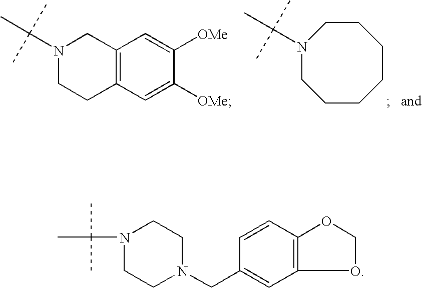

Particularly preferred within the general formula are embodiments in which R1 is at the para position and is —OH or —O—CH₂—CH₂—OH; those in which R2 is —H or —I; and those in which R3 is —O—CH₂—CH═CH₂. More particularly preferred is the compound in which R1 is at the para position and is —O—CH₂—CH₂—OH; R2 is —H; and R3 is —O—CH₂—CH═CH₂. Yet more particularly preferred is the compound in which R1 is at the para position and is —OH; R2 is —H; and R3 is —O—CH₂—CH═CH₂. Yet more particularly preferred is the compound in which R1 is at the para position and is —O—CH₂—CH₂—OH; R2 is —I; and R3 is —O—CH₂—CH═CH₂. Most particularly preferred is the compound in which R1 is at the para position and is —OH; R2 is —I; and R3 is —O—CH₂—CH═CH₂.

The present invention is to be understood to include all the various isomeric forms of the compounds of the general formula and mixtures thereof in any proportion. Thus, pure enantiomers, racemic mixtures and unequal mixtures of two enantiomers of compounds of the general formula are included within the present invention and may be used in accordance with the present invention. It should also be understood that all the diastereomeric forms possible are within the scope of the invention.

Other SPOX compounds that may be used in accordance with the present invention include those disclosed in U.S. Pat. No. 6,774,132, also incorporated herein by reference.

SPOX compounds that may be used in accordance with the present invention can be synthesized using synthetic chemistry techniques known in the art such as those disclosed in Lo et al., J. Am. Chem. Soc., 2004, 126, 16077-16086, incorporated herein by reference. Many useful methods for synthesis of oxindoles are reviewed by G. M. Karp in Org. Prep. Proced. Int. 1993, 25, 481-513, which is incorporated herein by reference. It is to be understood that certain functional groups may interfere with other reactants or reagents under the reaction conditions and therefore may need temporary protection. The use of protecting groups is described in ‘Protective Groups in Organic Synthesis’, 2nd edition, T. W. Greene & P. G. M. Wutz, Wiley-Interscience (1991).

In one embodiment, compositions of the invention comprise one or more SPOX compounds or pharmaceutically acceptable salts thereof. The term “non-DNA SPOX compound” or “SPOX compound” herein refers to any non-DNA compound having a spirooxindole ring that is capable of binding WRN. In one embodiment, the SPOX Compound is an agonist or partial agonist of WRN.

Depending on the process conditions the SPOX compound obtained may be either in neutral or salt form. Salt forms include hydrates and other solvates and also crystalline polymorphs. Both the free base and the salts of these end products are within the scope of the invention.

Acid addition salts of the SPOX compounds may in a manner known per se be transformed into the free base using basic agents such as alkali or by ion exchange. The free base obtained may also form salts with organic or inorganic acids.

In the preparation of acid addition salts, preferably such acids are used which form suitably pharmaceutically acceptable salts. Examples of such acids are hydrochloric acid, sulfuric acid, phosphoric acid, nitric acid, aliphatic acid, alicyclic carboxylic or sulfonic acids, such as formic acid, acetic acid, propionic acid, succinic acid, glycolic acid, lactic acid, malic acid, tartaric acid, citric acid, ascorbic acid, glucuronic acid, fumaric acid, maleic acid, hydroxymaleic acid, pyruvic acid, aspartic acid, glutamic acid, p-hydroxybenzoic acid, embonic acid, ethanesulfonic acid, hydroxyethanesulfonic acid, phenylacetic acid, mandelic acid, alogenbensenesulfonic acid, toluenesulfonic acid, galactaric acid, galacturonic acid or naphthalenesulfonic acid. All crystalline form polymorphs are within the scope of the invention.

SPOX compounds of the general formula wherein R3 is —OH are expected to have improved stability and water solubility relative to the corresponding esters, e.g. allyl esters. Pharmaceutically acceptable base addition salts of these SPOX compounds are provided and may be prepared by contacting the free acid form with a sufficient amount of the desired base to produce the salt in the conventional manner. The free acid form may be regenerated by contacting the salt form with an acid and isolating the free acid in the conventional manner. Pharmaceutically acceptable base addition salts are formed with metals or amines, such as alkali and alkali earth metals or organic amines. Examples of metals used as cations are sodium, potassium, calcium, magnesium and the like. Examples of suitable amines are amino acids such as lysine, choline, diethanolamine, ethylenediamine, N-methylglucamine and the like.

A SPOX compound can be present in a composition of the invention in any suitable amount, for example about 0.1 mg to about 1000 mg, about 0.5 mg to about 800 mg, about 1 mg to about 750 mg, or about 5 to about 500 mg. In another embodiment, a SPOX compound is present in a composition of the invention in an amount of about 1% to about 75%, about 5% to about 60%, or about 10% to about 50%, by weight of the composition.

### Dosage Forms

Compositions of the present invention can be formulated as dosage forms, for example solid, liquid, semi-solid or other dosage forms. In one embodiment, such compositions are in the form of discrete dose units or dosage units. The terms “dose unit” and/or “dosage unit” herein refer to a portion of a pharmaceutical composition that contains an amount of a therapeutic agent suitable for a single administration to provide a therapeutic effect. Such dosage units may be administered one to a small plurality (i.e. 1 to about 6) of times per day, or as many times as needed to elicit a therapeutic response. A particular dosage form can be selected to accommodate any desired frequency of administration to achieve a specified daily dose. Typically one dose unit, or a small plurality (i.e. up to about 6) of dose units, provides a sufficient amount of the active drug to result in the desired response or effect.

Compositions of the present invention may be prepared in the form of a dose unit or dose units suitable for oral, parenteral, transdermal, rectal, transmucosal, or topical administration. Parenteral administration includes, but is not limited to, intravenous, intraarterial, intraperitoneal, subcutaneous, intramuscular, intrathecal, and intraarticular.

The terms “oral administration” or “orally deliverable” herein include any form of delivery of a therapeutic agent or a composition thereof to a subject wherein the agent or composition is placed in the mouth of the subject, whether or not the agent or composition is swallowed. Thus “oral administration” includes buccal and sublingual as well as esophageal (e.g. inhalation) administration.

In still another embodiment, compositions of the present invention are formulated as rectal suppositories, which may contain suppository bases including, but not limited to, cocoa butter or glycerides.

Compositions of the present invention may also be formulated for inhalation, which may be in a form including, but not limited to, a solution, suspension, or emulsion that may be administered as a dry powder or in the form of an aerosol using a propellant, such as dichlorodifluoromethane or trichlorofluoromethane.

Compositions of the present invention may also be formulated for transdermal delivery, for example as a cream, ointment, lotion, paste, gel, medicated plaster, patch, or membrane. Such compositions can comprise any suitable excipients, for example penetration enhancers, etc.

Compositions of the present invention may also be formulated for parenteral administration including, but not limited to, by injection or continuous infusion. Formulations for injection may be in the form of suspensions, solutions, or emulsions in oily or aqueous vehicles. Such compositions may also be provided in a powder form for reconstitution with a suitable vehicle including, but not limited to, sterile, pyrogen-free water, WFI, etc.

Compositions of the present invention may also be formulated as a depot preparation, which may be administered by implantation or by intramuscular injection. Such compositions may be formulated with suitable polymeric or hydrophobic materials (as an emulsion in an acceptable oil, for example), ion exchange resins, or as sparingly soluble derivatives (as a sparingly soluble salt, for example).

Compositions of the present invention may also be formulated as a liposome preparation. Liposome preparations can comprise liposomes which penetrate the cells of interest or the stratum corneum and fuse with the cell membrane resulting in delivery of the contents of the liposome into the cell. For example, liposomes such as those described in U.S. Pat. No. 5,077,211 of Yarosh, U.S. Pat. No. 4,621,023 of Redziniak et al. or U.S. Pat. No. 4,508,703 of Redziniak et al. can be used. Where compositions of the invention are intended to target skin conditions, such compositions can be administered before, during, or after exposure of the skin of the mammal to UV or agents causing oxidative damage. Other suitable formulations can employ niosomes. Niosomes are lipid vesicles similar to liposomes, with membranes consisting largely of non-ionic lipids, some forms of which are effective for transporting compounds across the stratum corneum.

### Solid Dosage Forms

A composition of the invention can be in the form of solid dosage units such as tablets (e.g. suspension tablets, bite suspension tablets, rapid dispersion tablets, chewable tablets, effervescent tablets, bilayer tablets, etc), caplets, capsules (e.g. a soft or a hard gelatin capsule), powder (e.g. a packaged powder, a dispensable powder or an effervescent powder), lozenges, sachets, cachets, troches, pellets, granules, microgranules, encapsulated microgranules, powder aerosol formulations, or any other solid dosage form reasonably adapted for administration.

Tablets can be prepared according to any of the many relevant, well known pharmacy techniques. In one embodiment, tablets or other solid dosage forms can be prepared by processes that employ one or a combination of methods including, without limitation, (1) dry mixing, (2) direct compression, (3) milling, (4) dry or non-aqueous granulation, (5) wet granulation, or (6) fusion.

The individual steps in the wet granulation process of tablet preparation typically include milling and sieving of the ingredients, dry powder mixing, wet massing, granulation and final grinding. Dry granulation involves compressing a powder mixture into a rough tablet or “slug” on a heavy-duty rotary tablet press. The slugs are then broken up into granular particles by a grinding operation, usually by passage through an oscillation granulator. The individual steps include mixing of the powders, compressing (slugging) and grinding (slug reduction or granulation). Typically, no wet binder or moisture is involved in any of the steps.

In another embodiment, solid dosage forms can be prepared by mixing a SPOX compound with one or more pharmaceutical excipients to form a substantially homogeneous preformulation blend. The preformulation blend can then be subdivided and optionally further processed (e.g. compressed, encapsulated, packaged, dispersed, etc.) into any desired dosage forms.

Compressed tablets can be prepared by compacting a powder or granulation composition of the invention. The term “compressed tablet” generally refers to a plain, uncoated tablet suitable for oral ingestion, prepared by a single compression or by pre-compaction tapping followed by a final compression. Tablets of the present invention may be coated or otherwise compounded to provide a dosage form affording the advantage of improved handling or storage characteristics. In one embodiment, any such coating will be selected so as to not substantially delay onset of therapeutic effect of a composition of the invention upon administration to a subject. The term “suspension tablet” as used herein refers to a compressed tablet that rapidly disintegrates after placement in water.

### Liquid or Semi-Solid Dosage Forms

Suitable liquid dosage forms for a composition of the invention include solutions, aqueous or oily suspensions, elixirs, syrups, emulsions, liquid aerosol formulations, gels, creams, ointments, etc. Such compositions may also be formulated as a dry product for constitution with water or other suitable vehicle before use.

In one embodiment, liquid of semi-solid compositions, upon storage in a closed container maintained at either room temperature, refrigerated (e.g. about 5-10° C.) temperature, or freezing temperature for a period of about 1, 2, 3, 4, 5, 6, 7, 8, 9, 10, 11, or 12 months, exhibit at least about 90%, at least about 92.5%, at least about 95%, or at least about 97.5% of the original SPOX compound present therein.

### Pharmaceutical Excipients

Compositions of the invention can, if desired, include one or more pharmaceutically acceptable excipients. The term “excipient” herein means any substance, not itself a therapeutic agent, used as a carrier or vehicle for delivery of a therapeutic agent to a subject or added to a pharmaceutical composition to improve its handling or storage properties or to permit or facilitate formation of a unit dose of the composition. Excipients include, by way of illustration and not limitation, diluents, disintegrants, binding agents, adhesives, wetting agents, lubricants, glidants, surface modifying agents or surfactants, fragrances, suspending agents, emulsifying agents, nonaqueous vehicles, preservatives, antioxidants, adhesives, agents to adjust pH and osmolarity (e.g. buffering agents), preservatives, thickening agents, sweetening agents, flavoring agents, taste masking agents, colorants or dyes, penetration enhancers and substances added to improve appearance of the composition.

Excipients optionally employed in compositions of the invention can be solids, semi-solids, liquids or combinations thereof. Compositions of the invention containing excipients can be prepared by any known technique of pharmacy that comprises mixing an excipient with a drug or therapeutic agent.

Compositions of the invention optionally comprise one or more pharmaceutically acceptable diluents as excipients. Suitable diluents illustratively include, either individually or in combination, lactose, including anhydrous lactose and lactose monohydrate; starches, including directly compressible starch and hydrolyzed starches (e.g., Celutab™ and Emdex™); mannitol; sorbitol; xylitol; dextrose (e.g., Cerelose™ 2000) and dextrose monohydrate; dibasic calcium phosphate dihydrate; sucrose-based diluents; confectioner's sugar; monobasic calcium sulfate monohydrate; calcium sulfate dihydrate; granular calcium lactate trihydrate; dextrates; inositol; hydrolyzed cereal solids; amylose; celluloses including microcrystalline cellulose, food grade sources of α- and amorphous cellulose (e.g., Rexcel™) and powdered cellulose; calcium carbonate; glycine; bentonite; polyvinylpyrrolidone; and the like. Such diluents, if present, constitute in total about 5% to about 99%, about 10% to about 85%, or about 20% to about 80%, of the total weight of the composition. Any diluent or diluents selected preferably exhibit suitable flow properties and, where tablets are desired, compressibility.

The use of extragranular microcrystalline cellulose (that is, microcrystalline cellulose added to a wet granulated composition after a drying step) can be used to improve hardness (for tablets) and/or disintegration time.

Compositions of the invention optionally comprise one or more pharmaceutically acceptable disintegrants as excipients, particularly for tablet, capsule or other solid formulations. Suitable disintegrants include, either individually or in combination, starches, including sodium starch glycolate (e.g., Explotab™ of PenWest) and pregelatinized corn starches (e.g., National™ 1551, National™ 1550, and Colocorn™ 1500), clays (e.g., Veegum™ HV), celluloses such as purified cellulose, microcrystalline cellulose, methylcellulose, carboxymethylcellulose and sodium carboxymethylcellulose, croscarmellose sodium (e.g., Ac-Di-Sol™ of FMC), alginates, crospovidone, and gums such as agar, guar, xanthan, locust bean, karaya, pectin and tragacanth gums.

Disintegrants may be added at any suitable step during the preparation of the composition, particularly prior to a granulation step or during a lubrication step prior to compression. Such disintegrants, if present, constitute in total about 0.2% to about 30%, about 0.2% to about 10%, or about 0.2% to about 5%, of the total weight of the composition.

Compositions of the invention optionally comprise one or more pharmaceutically acceptable binding agents or adhesives as excipients, particularly for tablet formulations. Such binding agents and adhesives preferably impart sufficient cohesion to the powder being tableted to allow for normal processing operations such as sizing, lubrication, compression and packaging, but still allow the tablet to disintegrate and the composition to be absorbed upon ingestion. Suitable binding agents and adhesives include, either individually or in combination, acacia; tragacanth; sucrose; gelatin; glucose; starches such as, but not limited to, pregelatinized starches (e.g., National™ 1511 and National™ 1500); celluloses such as, but not limited to, methylcellulose and carmellose sodium (e.g., Tylose™); alginic acid and salts of alginic acid; magnesium aluminum silicate; PEG; guar gum; polysaccharide acids; bentonites; povidone, for example povidone K-15, K-30 and K-29/32; polymethacrylates; HPMC; hydroxypropylcellulose (e.g., Klucel™); and ethylcellulose (e.g., Ethocel™). Such binding agents and/or adhesives, if present, constitute in total about 0.5% to about 25%, about 0.75% to about 15%, or about 1% to about 10%, of the total weight of the composition.

Compositions of the invention optionally comprise one or more pharmaceutically acceptable wetting agents as excipients. Non-limiting examples of surfactants that can be used as wetting agents in compositions of the invention include quaternary ammonium compounds, for example benzalkonium chloride, benzethonium chloride and cetylpyridinium chloride, dioctyl sodium sulfosuccinate, polyoxyethylene alkylphenyl ethers, for example nonoxynol 9, nonoxynol 10, and octoxynol 9, poloxamers (polyoxyethylene and polyoxypropylene block copolymers), polyoxyethylene fatty acid glycerides and oils, for example polyoxyethylene (8) caprylic/capric mono- and diglycerides (e.g., Labrasol™ of Gattefossé), polyoxyethylene (35) castor oil and polyoxyethylene (40) hydrogenated castor oil; polyoxyethylene alkyl ethers, for example polyoxyethylene (20) cetostearyl ether, polyoxyethylene fatty acid esters, for example polyoxyethylene (40) stearate, polyoxyethylene sorbitan esters, for example polysorbate 20 and polysorbate 80 (e.g., Tween™ 80 of ICI), propylene glycol fatty acid esters, for example propylene glycol laurate (e.g., Lauroglycol™ of Gattefossé), sodium lauryl sulfate, fatty acids and salts thereof, for example oleic acid, sodium oleate and triethanolamine oleate, glyceryl fatty acid esters, for example glyceryl monostearate, sorbitan esters, for example sorbitan monolaurate, sorbitan monooleate, sorbitan monopalmitate and sorbitan monostearate, tyloxapol, and mixtures thereof. Such wetting agents, if present, constitute in total about 0.25% to about 15%, about 0.4% to about 10%, or about 0.5% to about 5%, of the total weight of the composition.

Compositions of the invention optionally comprise one or more pharmaceutically acceptable lubricants (including anti-adherents and/or glidants) as excipients. Suitable lubricants include, either individually or in combination, glyceryl behapate (e.g., Compritol™ 888); stearic acid and salts thereof, including magnesium (magnesium stearate), calcium and sodium stearates; hydrogenated vegetable oils (e.g., Sterotex™); colloidal silica; talc; waxes; boric acid; sodium benzoate; sodium acetate; sodium fumarate; sodium chloride; DL-leucine; PEG (e.g., Carbowax™ 4000 and Carbowax™ 6000); sodium oleate; sodium lauryl sulfate; and magnesium lauryl sulfate. Such lubricants, if present, constitute in total about 0.1% to about 10%, about 0.2% to about 8%, or about 0.25% to about 5%, of the total weight of the composition.

Suitable anti-adherents include talc, cornstarch, DL-leucine, sodium lauryl sulfate and metallic stearates. Talc is an anti-adherent or glidant used, for example, to reduce formulation sticking to equipment surfaces and also to reduce static in the blend. One or more anti-adherents, if present, constitute about 0.1% to about 10%, about 0.25% to about 5%, or about 0.5% to about 2%, of the total weight of the composition.

Glidants can be used to promote powder flow of a solid formulation. Suitable glidants include colloidal silicon dioxide, starch, talc, tribasic calcium phosphate, powdered cellulose and magnesium trisilicate. Colloidal silicon dioxide is particularly preferred.

Compositions of the present invention can comprise one or more anti-foaming agents. Simethicone is an illustrative anti-foaming agent. Anti-foaming agents, if present, constitute about 0.001% to about 5%, about 0.001% to about 2%, or about 0.001% to about 1%, of the total weight of the composition.

Illustrative antioxidants for use in the present invention include, but are not limited to, butylated hydroxytoluene, butylated hydroxyanisole, potassium metabisulfite, and the like. One or more antioxidants, if desired, are typically present in a composition of the invention in an amount of about 0.01% to about 2.5%, for example about 0.01%, about 0.05%, about 0.1%, about 0.5%, about 1%, about 1.5%, about 1.75%, about 2%, about 2.25%, or about 2.5%, by weight.

In various embodiments, compositions of the invention can comprise a preservative. Suitable preservatives include, but are not limited to, benzalkonium chloride, methyl, ethyl, propyl or butylparaben, benzyl alcohol, phenylethyl alcohol, benzethonium, methyl or propyl p-hydroxybenzoate and sorbic acid or combinations thereof. Typically, the optional preservative is present in an amount of about 0.01% to about 0.5% or about 0.01% to about 2.5%, by weight.

In one embodiment, compositions of the invention optionally comprise a buffering agent. Buffering agents include agents that reduce pH changes. Illustrative classes of buffering agents for use in various embodiments of the present invention comprise a salt of a Group IA metal including, for example, a bicarbonate salt of a Group IA metal, a carbonate salt of a Group IA metal, an alkaline or alkali earth metal buffering agent, an aluminum buffering agent, a calcium buffering agent, a sodium buffering agent, or a magnesium buffering agent. Suitable buffering agents include carbonates, phosphates, bicarbonates, citrates, borates, acetates, phthalates, tartrates, succinates of any of the foregoing, for example sodium or potassium phosphate, citrate, borate, acetate, bicarbonate and carbonate.

Non-limiting examples of suitable buffering agents include aluminum, magnesium hydroxide, aluminum glycinate, calcium acetate, calcium bicarbonate, calcium borate, calcium carbonate, calcium citrate, calcium gluconate, calcium glycerophosphate, calcium hydroxide, calcium lactate, calcium phthalate, calcium phosphate, calcium succinate, calcium tartrate, dibasic sodium phosphate, dipotassium hydrogen phosphate, dipotassium phosphate, disodium hydrogen phosphate, disodium succinate, dry aluminum hydroxide gel, magnesium acetate, magnesium aluminate, magnesium borate, magnesium bicarbonate, magnesium carbonate, magnesium citrate, magnesium gluconate, magnesium hydroxide, magnesium lactate, magnesium metasilicate aluminate, magnesium oxide, magnesium phthalate, magnesium phosphate, magnesium silicate, magnesium succinate, magnesium tartrate, potassium acetate, potassium carbonate, potassium bicarbonate, potassium borate, potassium citrate, potassium metaphosphate, potassium phthalate, potassium phosphate, potassium polyphosphate, potassium pyrophosphate, potassium succinate, potassium tartrate, sodium acetate, sodium bicarbonate, sodium borate, sodium carbonate, sodium citrate, sodium gluconate, sodium hydrogen phosphate, sodium hydroxide, sodium lactate, sodium phthalate, sodium phosphate, sodium polyphosphate, sodium pyrophosphate, sodium sesquicarbonate, sodium succinate, sodium tartrate, sodium tripolyphosphate, synthetic hydrotalcite, tetrapotassium pyrophosphate, tetrasodium pyrophosphate, tripotassium phosphate, trisodium phosphate, and trometamol. (Based in part upon the list provided in The Merck Index, Merck & Co. Rahway, N.J. (2001)). Furthermore, combinations or mixtures of any two or more of the above mentioned buffering agents can be used in the pharmaceutical compositions described herein. One or more buffering agents, if desired, are present in compositions of the invention in an amount of about 0.01% to about 5% or about 0.01% to about 3%, by weight.

In various embodiments, compositions the invention may include one or more agents that increase viscosity. Illustrative agents that increase viscosity include, but are not limited to, methylcellulose, carboxymethylcellulose sodium, ethylcellulose, carrageenan, carbopol, and/or combinations thereof. Typically, one or more viscosity increasing agents, if desired, are present in compositions of the invention in an amount of about 0.1% to about 10%, or about 0.1% to about 5%, by weight.

In various embodiments, compositions of the invention comprise an “organoleptic agent” to improve the organoleptic properties of the composition. The term “organoleptic agent” herein refers to any excipient that can improve the flavor or odor of or help mask a disagreeable flavor or odor of a composition of the invention. Such agents include sweeteners, flavoring agents and/or taste masking agents. Suitable sweeteners and/or flavoring agents include any agent that sweetens or provides flavor to a pharmaceutical composition. Optional organoleptic agents are typically present in a composition of the invention in an amount of about 0.1 mg/ml to about 10 mg/ml, about 0.5 mg/ml to 5 mg/ml or about 1 mg/ml.

Illustrative sweeteners or flavoring agents include, without limitation, acacia syrup, anethole, anise oil, aromatic elixir, benzaldehyde, benzaldehyde elixir, cyclodextrins, caraway, caraway oil, cardamom oil, cardamom seed, cardamom spirit, cardamom tincture, cherry juice, cherry syrup, cinnamon, cinnamon oil, cinnamon water, citric acid, citric acid syrup, clove oil, cocoa, cocoa syrup, coriander oil, dextrose, eriodictyon, eriodictyon fluidextract, eriodictyon syrup, aromatic, ethylacetate, ethyl vanillin, fennel oil, ginger, ginger fluidextract, ginger oleoresin, dextrose, glucose, sugar, maltodextrin, glycerin, glycyrrhiza, glycyrrhiza elixir, glycyrrhiza extract, glycyrrhiza extract pure, glycyrrhiza fluid extract, glycyrrhiza syrup, honey, iso-alcoholic elixir, lavender oil, lemon oil, lemon tincture, mannitol, methyl salicylate, nutmeg oil, orange bitter, elixir, orange bitter, oil, orange flower oil, orange flower water, orange oil, orange peel, bitter, orange peel sweet, tincture, orange spirit, orange syrup, peppermint, peppermint oil, peppermint spirit, peppermint water, phenylethyl alcohol, raspberry juice, raspberry syrup, rosemary oil, rose oil, rose water, stronger, saccharin, saccharin calcium, saccharin sodium, sarsaparilla syrup, sarsaparilla, sorbitol solution, spearmint, spearmint oil, sucrose, sucralose, syrup, thyme oil, tolu balsam, tolu balsam syrup, vanilla, vanilla tincture, vanillin, wild cherry syrup, or combinations thereof.

Illustrative taste masking agents include, but are not limited to, cyclodextrins, cyclodextrins emulsions, cyclodextrins particles, cyclodextrins complexes, or combinations thereof.

Illustrative suspending agents include, but are not limited to, sorbitol syrup, methyl cellulose, glucose/sugar syrup, gelatin, hydroxyethylcellulose, carboxymethyl cellulose, aluminum stearate gel, and hydrogenated edible fats.

Illustrative emulsifying agents include, but are not limited to, lecithin, sorbitan monooleate, and acacia. Nonaqueous vehicles include, but are not limited to, edible oils, almond oil, fractionated coconut oil, oily esters, propylene glycol, and ethyl alcohol.

The foregoing excipients can have multiple roles as is known in the art. For example, starch can serve as a filler as well as a disintegrant. The classification of excipients above is not to be construed as limiting in any manner

In one embodiment, a composition of the invention comprises a nucleus-specific targeting or carrier, for example a nuclear protein. The term “nucleus-specific carrier or targeting” herein refers to molecules capable of transporting a molecule to the nucleus of a cell. Such molecules include but are not limited to endothelial protein C receptor, transcription factors nuclear localization signal of SV-40 virus, SV-40 large T antigen, nuclear localization of HIV type 1 TAT.

### Administration

Compositions of the invention may be used alone or in combination with other modalities to treat and/or prevent conditions associated with failure of growth arrest, apoptosis or proliferative senescence. Representative examples of such conditions include, but are not limited to, hyperproliferative diseases, such as cancer and the benign growth of cells beyond a normal range as, for example, keratinocytes in psoriasis or fibroblast hypertrophic scars and keloids, or certain subsets of lymphocytes in the case of various autoimmune disorders. Cancers to be treated by these methods arise in various cell types and organs of the body, for example, neuroblastoma, retinoblastoma, glioblastoma, tumors of the respiratory tract, bronchogenic carcinoma, large cell carcinoma, tumors of the urogenital tract, adenocarcinoma, papillary carcinoma, hepatocellular carcinoma, cervical cancer, lymphoma such as B cell, Hodgkin's, Non-Hodgkins, large cell, or diffuse lymphoma, osteosarcoma, squamous cell carcinoma, basal cell carcinoma, melanoma and other cancers arising in the skin, and tumors of blood cells and related cells including acute and chronic leukemia. Compositions of the invention can also be used to treat and/or prevent cancers of the breast, lung, liver, prostate, pancreas, ovaries, bladder, uterus, colon, brain, esophagus, stomach, and thyroid. Compositions of the invention may also be used to induce tanning, to promote cellular differentiation and for immunosuppression, for example in relation to organ transplants.

In one embodiment, a composition of the invention comprises SPOX-1 and is used to treat a cancer selected from the group consisting of lymphoma, glioblastoma, osteosarcoma, melanoma, leukemia, and carcinomas of the skin, breast, lung, liver, prostate, cervix, pancreas, ovary, bladder, uterus, colon, esophagus, stomach, and thyroid.

The term “treat” or “treatment” as used herein refers to any treatment of a disorder or disease associated with failure of growth arrest, apoptosis or proliferative senescence, and includes, but is not limited to, inhibiting the disorder or disease arresting the development of the disorder or disease; relieving the disorder or disease, for example, causing regression of the disorder or disease; or relieving the condition caused by the disease or disorder, relieving the symptoms of the disease or disorder.

The term “prevent” or “prevention,” in relation to a disorder or disease associated with failure of growth arrest, apoptosis or proliferative senescence, means preventing the onset of disorder or disease development if none had occurred, or preventing further disorder or disease development if the disorder or disease was already present. For example, compositions of the present invention may be used to prevent the recurrence of tumors. Recurrence of tumors may occur because of residual microscopic groups or nests of tumor cells which subsequently expand into clinically detectable tumors.

Compositions of the present invention may be administered in any manner including, but not limited to, orally, parenterally, sublingually, transdermally, rectally, transmucosally, topically, via inhalation, via buccal administration, or combinations thereof. Parenteral administration includes, but is not limited to, intravenous, intraarterial, intraperitoneal, subcutaneous, intramuscular, intrathecal, intraarticular, intracisternal and intraventricular.

A therapeutically effective amount of the composition required for use in therapy varies with the nature of the condition being treated, the length of time that activity is desired, and the age and the condition of the patient to be treated, among other factors, and is ultimately determined by the attendant physician. In general, however, doses employed for human treatment typically are in the range of about 0.001 mg/kg to about 200 mg/kg per day, for example about 1 μg/kg to about 1 mg/kg per day or about 1 μg/kg to about 100 μg/kg per day. The desired dose may be conveniently administered in a single dose, or as multiple doses administered at appropriate intervals, for example as two, three, four or more subdoses per day.

Illustratively, a composition of the invention may be administered to a subject to provide the subject with a SPOX compound in an amount of about 1 μg/kg to about 1 mg/kg body weight, for example about 1 μg/kg, about 25 μg/kg, about 50 μg/kg, about 75 μg/kg, about 100 μg/kg, about 125 μg/kg, about 150 μg/kg, about 175 μg/kg, about 200 μg/kg, about 225 μg/kg, about 250 μg/kg, about 275 μg/kg, about 300 μg/kg, about 325 μg/kg, about 350 μg/kg, about 375 μg/kg, about 400 μg/kg, about 425 μg/kg, about 450 μg/kg, about 475 μg/kg, about 500 μg/kg, about 525 μg/kg, about 550 μg/kg, about 575 μg/kg, about 600 μg/kg, about 625 μg/kg, about 650 μg/kg, about 675 μg/kg, about 700 μg/kg, about 725 μg/kg, about 750 μg/kg, about 775 μg/kg, about 800 μg/kg, about 825 μg/kg, about 850 μg/kg, about 875 μg/kg, about 900 μg/kg, about 925 μg/kg, about 950 μg/kg, about 975 μg/kg or about 1 mg/kg body weight.

Those skilled in the art will readily appreciate that numerous other embodiments, modifications and equivalents are contemplated and encompassed by the disclosure of the present invention.

All patents, patent applications and publications referenced herein are hereby incorporated by reference herein to the fullest extent allowed under the law.

### EXAMPLES

The following examples illustrate various aspects of the present invention and are not to be construed as limiting the scope of the invention in any manner whatsoever.

### Example 1

### Phosphorylation of H2AX, Induction of Growth Arrest by SPOX in Human Fibroblasts

Oligonucleotides homologous to the telomere overhang repeat sequence (T-oligos) were previously shown to induce growth arrest, apoptosis and promote differentiation in U.S. patent application Ser. No. 10/122,633, filed Apr. 12, 2002, which is incorporated herein by reference. In the following experiments, T-oligos were compared to SPOX to determine whether the non-DNA small molecules have the same effect.

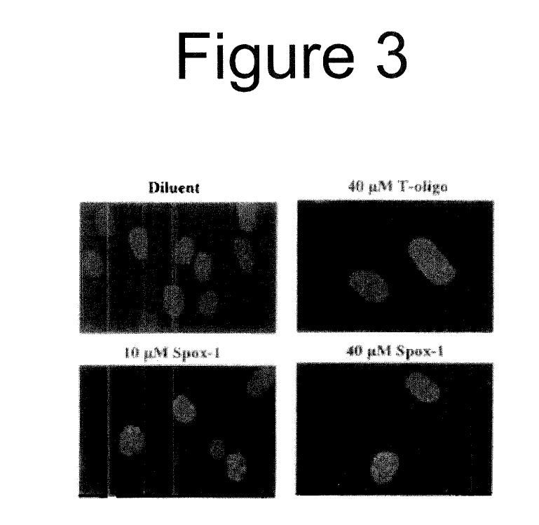

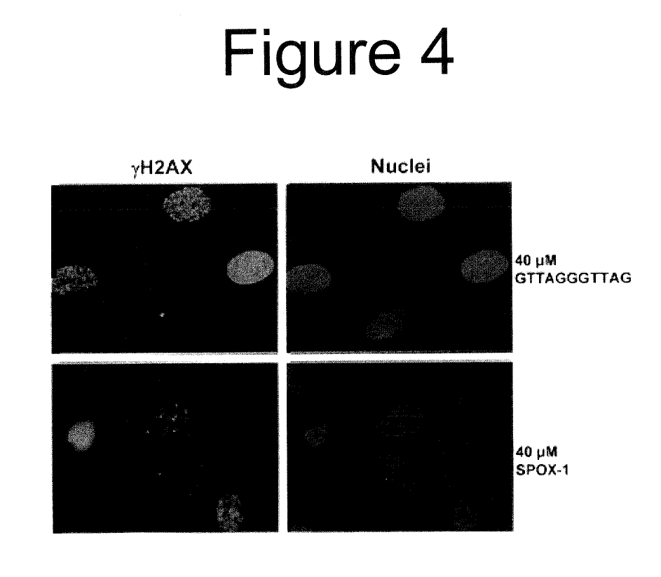

Phosphorylation of Histone H2AX (forming γH2AX) is a marker for DNA damage in many settings and is also observed at telomeres when cells enter senescence. As shown in FIG. 3 and FIG. 4, SPOX-1 and (11mer: 5′ GTTAGGGTTAG 3′; SEQ ID NO: 1) T-oligo both induce γH2AX expression at similar concentrations in human fibroblasts. In both experiments, cells were incubated for 48 hours in the presence of SPOX-1 or 11mer T-oligo before processing for immunofluorescent microscopy.

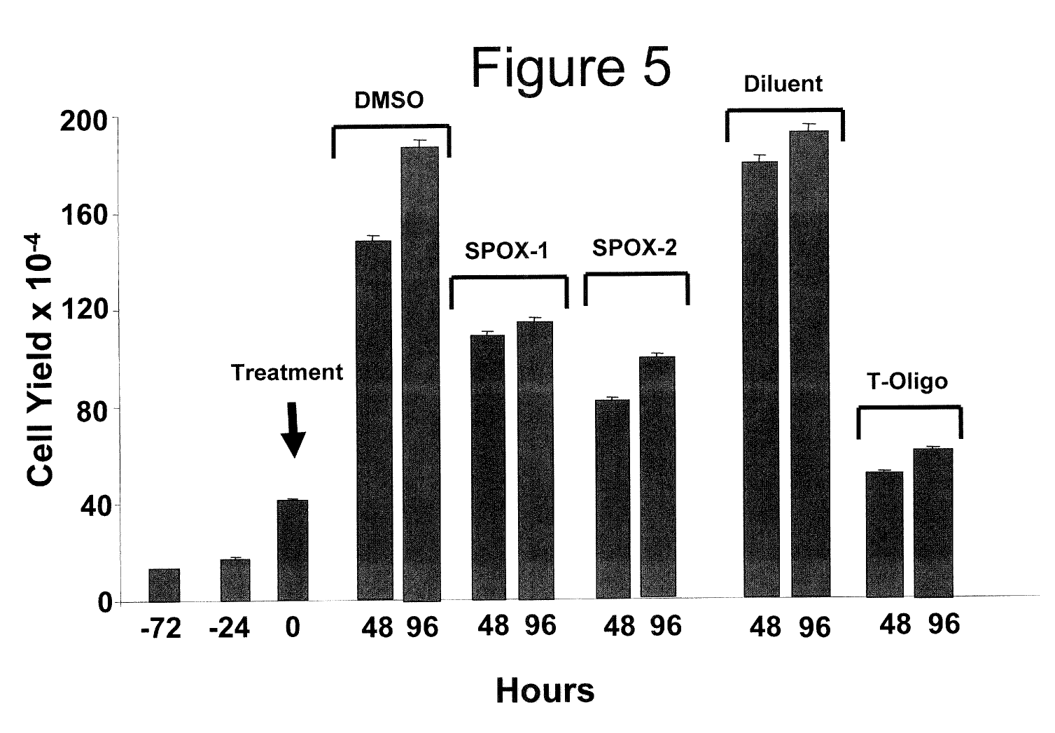

The effect of SPOX compounds on growth of human fibroblasts was tested using cultures of newborn fibroblast cells. Briefly, 20,000 cells were plated per 35 mm dish. Medium containing either diluent (dimethyl sulfoxide (DMSO)) alone, SPOX-1 (40 μM) SPOX-2 (40 μM) or 11mer T-oligo (SEQ ID NO:1) was introduced at 96 hours and growth was determined 2 and 4 days after plating. Both SPOX-1 and SPOX-2 inhibited growth of human fibroblast cells as shown in FIG. 5.

### Example 2

### SPOX Compounds Induce Apoptosis and Growth Arrest and Activate ATM in Human Melanoma Cells

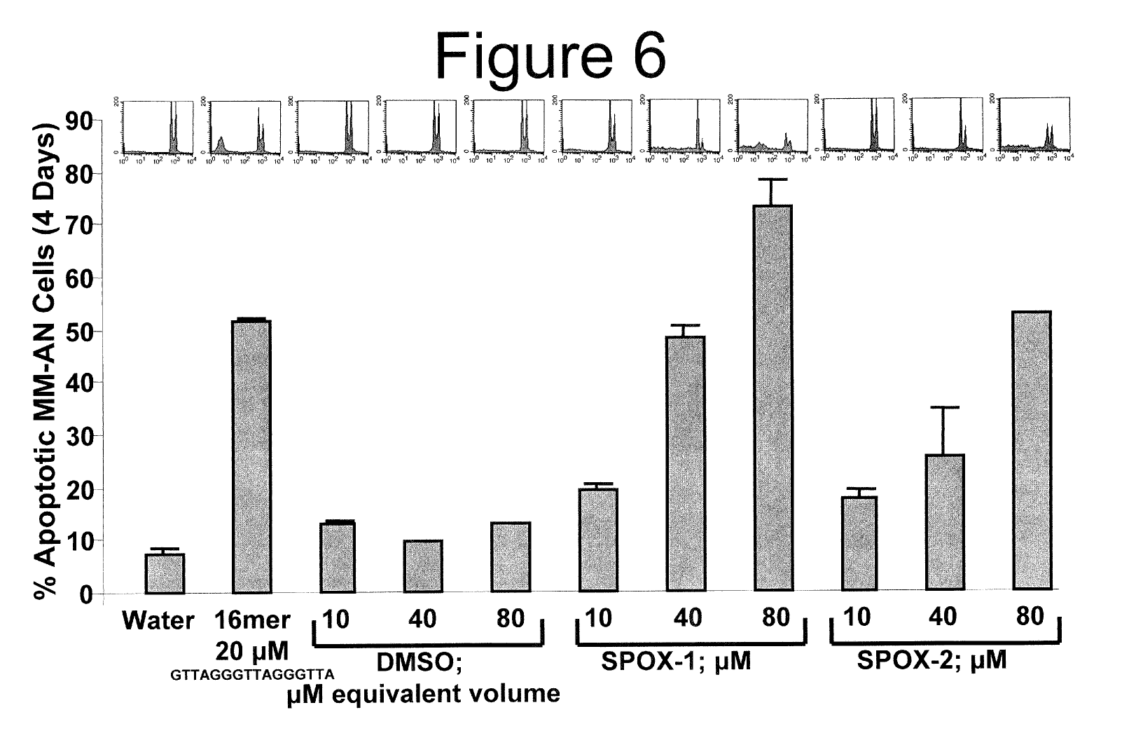

Cultures of MM-AN human melanoma cells were treated with diluent (water or DMSO) alone, a T-oligo (16mer: 5′ GTTAGGGTTAGGGTTA 3′; SEQ ID NO: 2), SPOX-1, or SPOX-2 for 96 hours, then collected and processed for FACS analysis. One concentration of the T-oligo (20 μM) and three concentrations of each SPOX compound (10 μM, 40 μM and 80 μM) were tested. Results are shown in FIG. 6. Each bar is the mean of triplicate dishes +/− standard error of the mean (SEM). FACS profiles are depicted at the top of the figure. As can be seen, both SPOX compounds induced apoptosis with SPOX-1 having a greater effect. The results demonstrate that each compound was able to mimic the effect of 16mer T-oligo on induction of apoptosis in the melanoma cells.

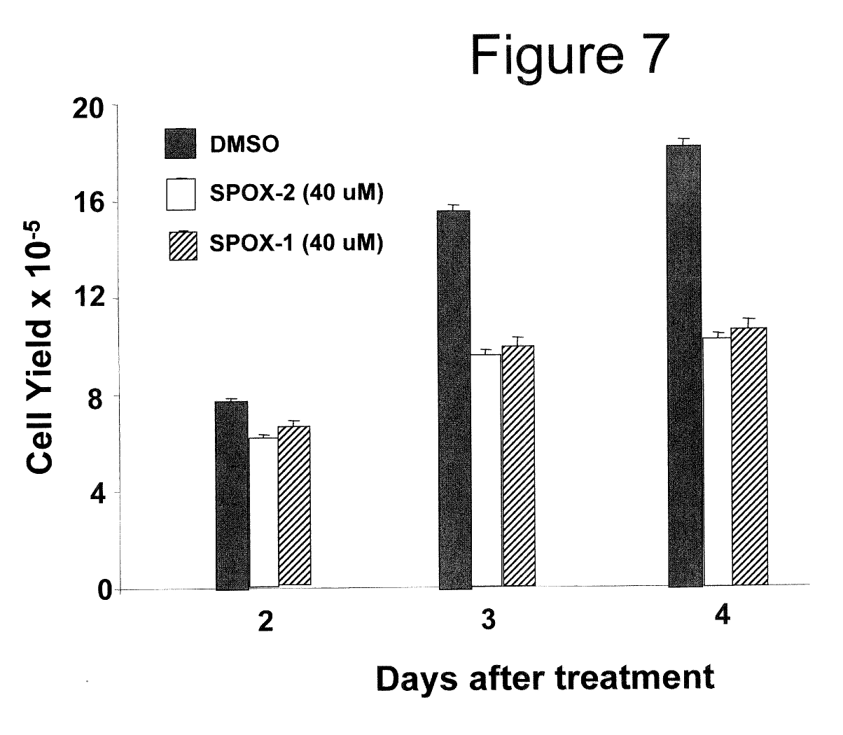

The effect of SPOX compounds on growth of human melanoma cells was tested using cultures of MM-AN cells. Briefly, 20,000 MM-AN cells were plated per 35 mm dish. Medium containing either diluent (DMSO) alone, SPOX-1 or SPOX-2 (40 μM) was introduced at 96 hours and growth was determined 2, 3 and 4 days after treatment. Both SPOX-1 and SPOX-2 inhibited growth of MM-AN cells as shown in FIG. 7.

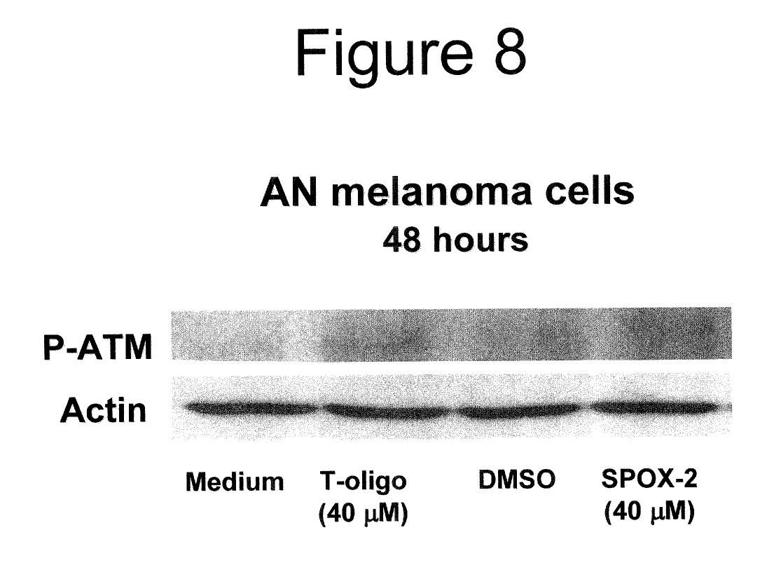

Phosphorylation of ataxia telanglectasia mutated (ATM) kinase at serine 1981 is a marker for DNA damage and has been demonstrated to occur following exposure of the telomere 3′ overhang. Phosphorylated ATM in turn phosphorylates, and thus activates, p53. MM-AN cells were treated with diluent (medium or DMSO) alone, 40 μM of 11mer T-oligo (SEQ ID NO: 2) or 40 μM of SPOX-2 for 48 hours then collected and analyzed by Western blot using an antibody against ATM phosphoserine 1981. As shown in FIG. 8, SPOX-2 and 11mer T-oligo both induce phosphorylation of ATM at similar concentrations in MM-AN cells.

### Example 3

### SPOX Compounds Induce Growth Arrest and Apoptosis in Human Breast Cancer Cells

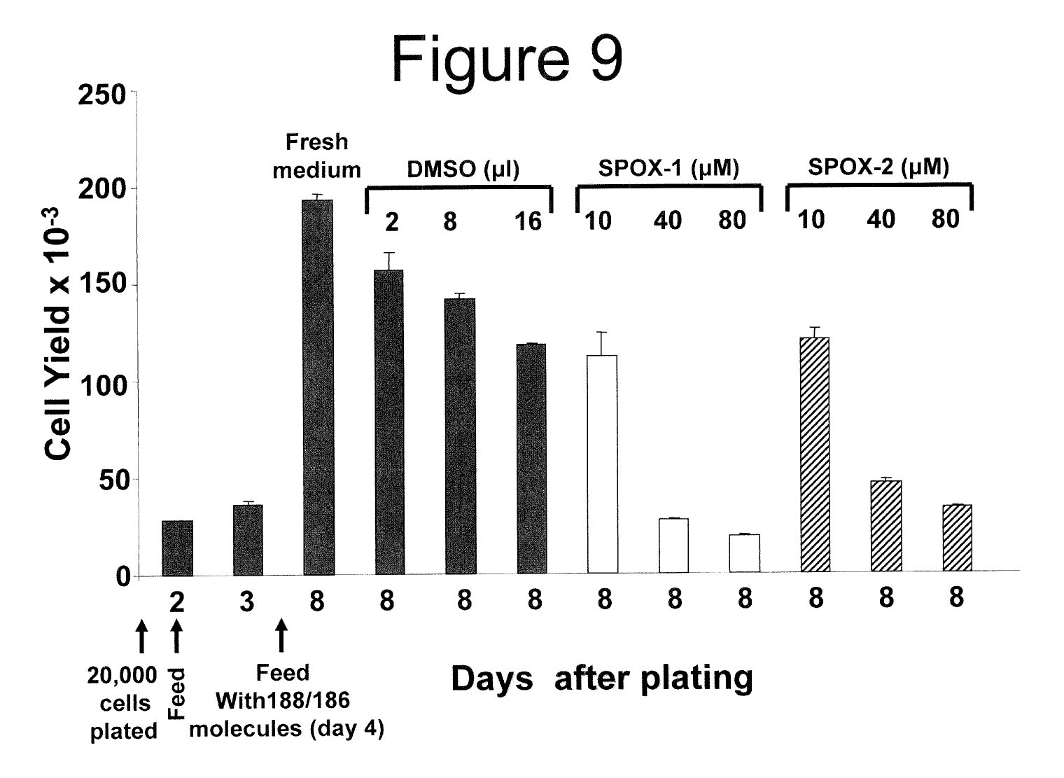

The effect of SPOX compounds on growth and apoptosis of human breast cancer cells was tested using cultures of MCF-7 cells. The effect on cell growth was tested by plating 20,000 cells per 35 mm dish. Medium containing either diluent alone (DMSO), SPOX-1 or SPOX-2 (10 μM, 40 μM and 80 μM) was introduced at 96 hours and growth was determined 8 days after plating. Both SPOX-1 and SPOX-2 inhibited growth of MCF-7 cells in a dose-dependent manner with the higher concentrations of 40 μM and 80 μM leading to nearly complete inhibition of growth as shown in FIG. 9.

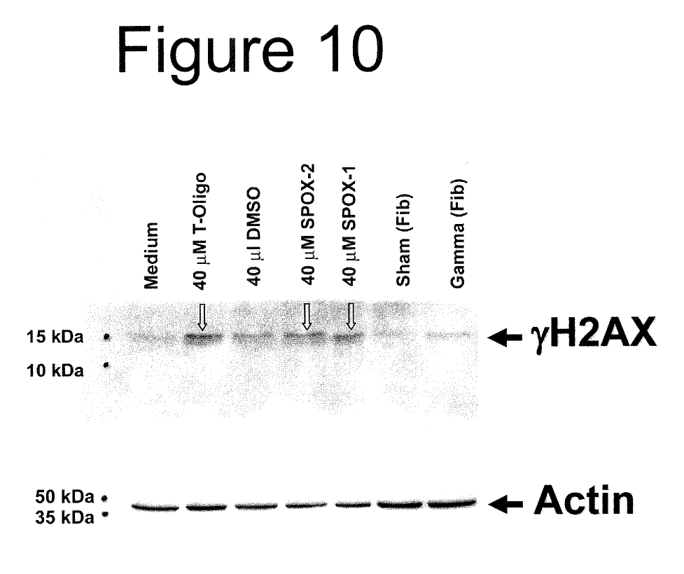

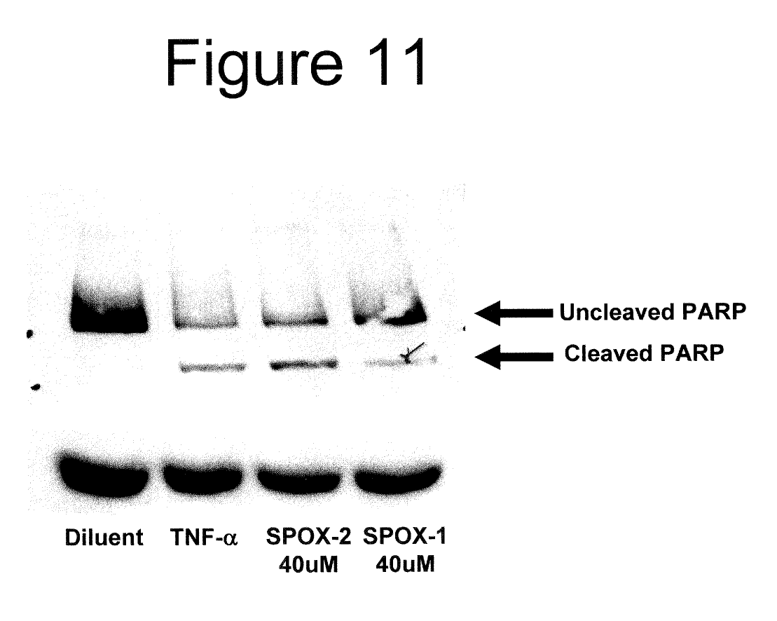

Phosphorylation of Histone H2AX (forming γH2AX) was assessed as a marker for DNA damage. Apoptosis was measured by western blot analysis to show cleavage of poly(ADP-ribose) polymerase (PARP). PARP is a substrate for caspase-3, an enzyme that mediates apoptosis, and thus cleavage of PARP by caspase-3 is indicative of ongoing apoptosis. FIG. 10 shows that both SPOX-1 and SPOX-2 cause phosphorylation of the histone protein H2AX, forming γH2AX, comparably to the positive control 16mer T-oligo (SEQ ID NO: 2). FIG. 11 shows that both SPOX-1 and SPOX-2 also cause PARP cleavage (mediated through caspase-3) comparably to the positive control compound TNF-α, known to cause apoptosis through this pathway. Thus, both SPOX compounds induce apoptosis and formation of γH2AX, as a marker of DNA damage-like responses, in the MCF-7 breast cancer cell line.

### Example 4

### SPOX-1 Induces Apoptosis Independent of WRN

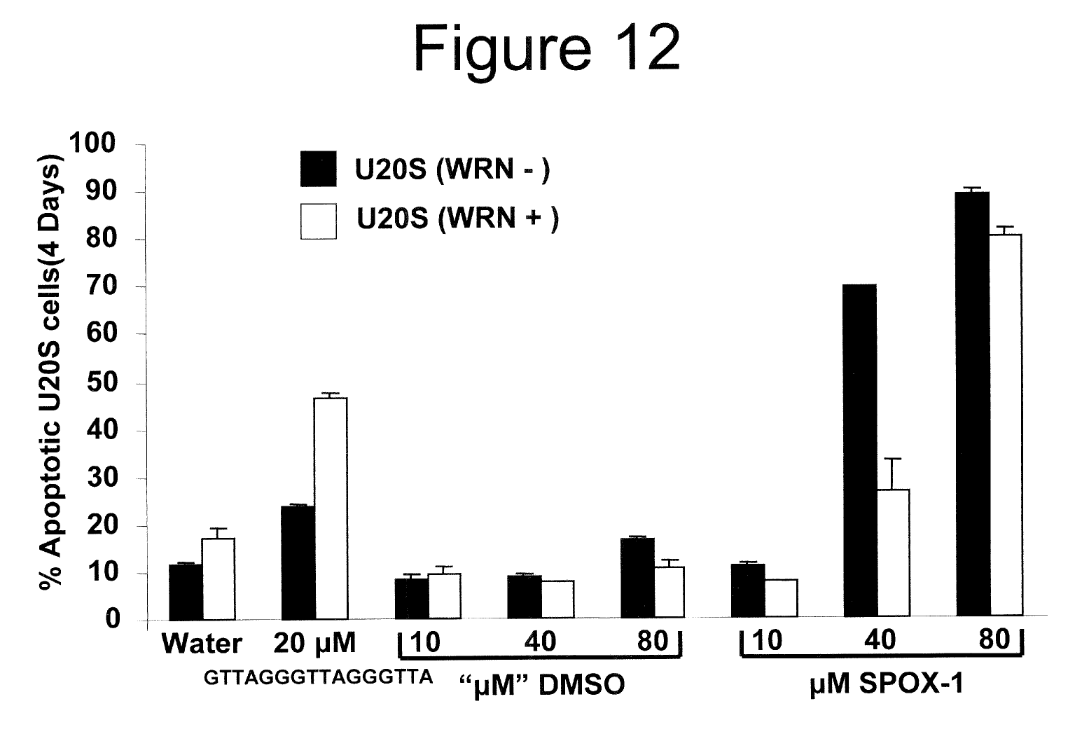

Cultures of WRN+ and WRN− U20S human osteosarcoma cells were treated with either diluent (water or DMSO) alone, 16mer T-oligo (SEQ ID NO: 2) or SPOX-1 for 96 hours, then collected and processed for FACS analysis. One concentration of the 16mer T-oligo was tested, 20 μM, and three concentrations of SPOX-1 were tested, 10 μM, 40 μM and 80 μM. FIG. 12 shows the results wherein 16mer T-oligo was less effective in inducing apoptosis in WRN− than in U20S cells with intact WRN. In contrast, while SPOX-1 also induced apoptosis in WRN+ U20S cells in a dose-dependent manner, apoptosis was also seen in the WRN− U20S cells at the 40 μM and 80 μM concentrations, suggesting that SPOX-1 may be capable of inducing apoptosis through WRN-dependent and WRN-independent pathways. Alternatively, because WRN− U20S cells comprise detectable, albeit greatly reduced, concentrations of WRN, it is possible that SPOX-1 can more efficiently utilize the lesser amount of WRN in these cells than can the T-oligo, perhaps through interaction with one or more components of the MRN complex in addition to WRN.

### Example 5

### SPOX Compounds Induce Melanogenesis in Human Skin

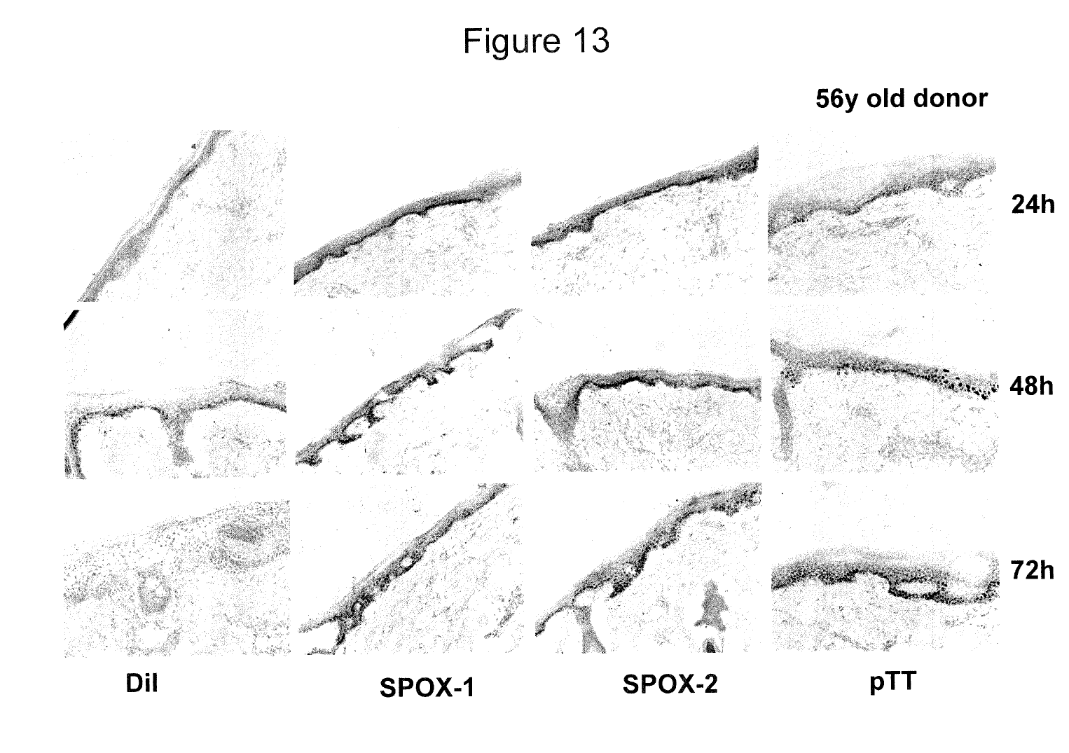

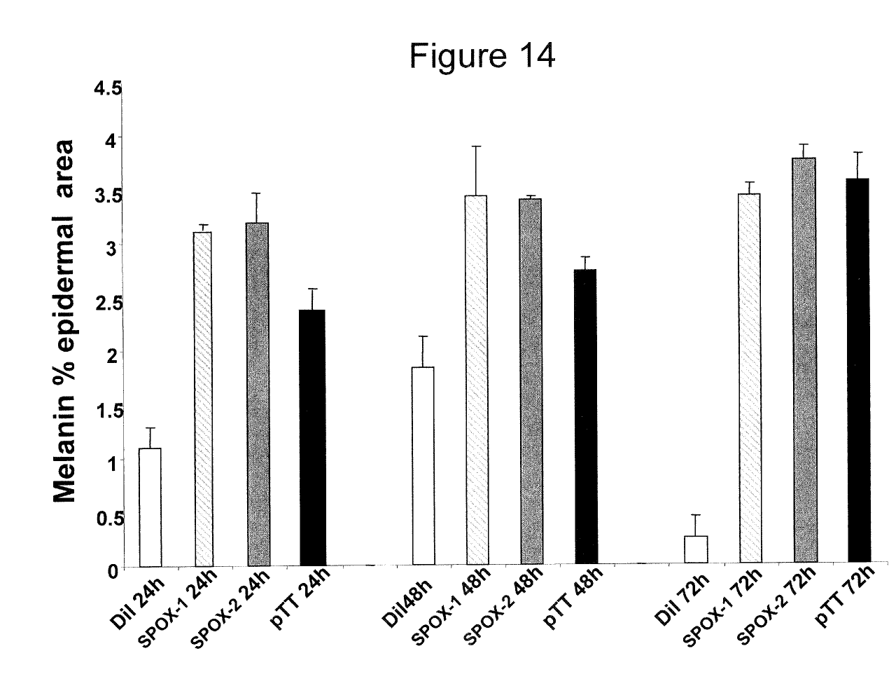

Normal facial skin removed from a 56 year old Caucasian woman at the time of facelift was carefully cut into fragments approximately 5×5 mm. The fragments were placed in tissue culture dishes in standard culture medium, under conditions known to maintain the fragments in good condition, responsive to physiologic stimuli, for at least one week (Arad et al, FASEB J. Jul. 28, 2006. [Epub]). Fragments were arranged with at least 3 per 35 mm dish and after 48 hours, one dish each was provided with fresh medium containing diluent (DMSO) alone (Dil) or SPOX-1 or SPOX-2 each at 80 uM or thymidine dinucleotide (pTT) at 100 uM as a positive control. After an additional 24, 48, and 72 hours, one tissue fragment was removed at each time from each of the dishes representing different treatment groups, snap frozen and stained with Fontana Masson stain to demonstrate melanin (FIG. 13) and subjected to computer-assisted image analysis to determine the percent epidermis occupied by melanin (FIG. 14). The 72 hour diluent (Dil) control sample was judged to be bacterially contaminated, affecting both the overall histology and the apparent melanin content, which based on earlier experiments would be expected to be the same as at 24 and 48 hours. All other samples were believed to be healthy and to accurately represent the response to treatment. As can be seen from FIG. 13 and FIG. 14, both SPOX compounds caused tanning, comparable to pTT.

### Example 6

### SPOX Compounds Reduce Survivin Expression and Induce Growth Arrest in Human Lung Cancer Cells

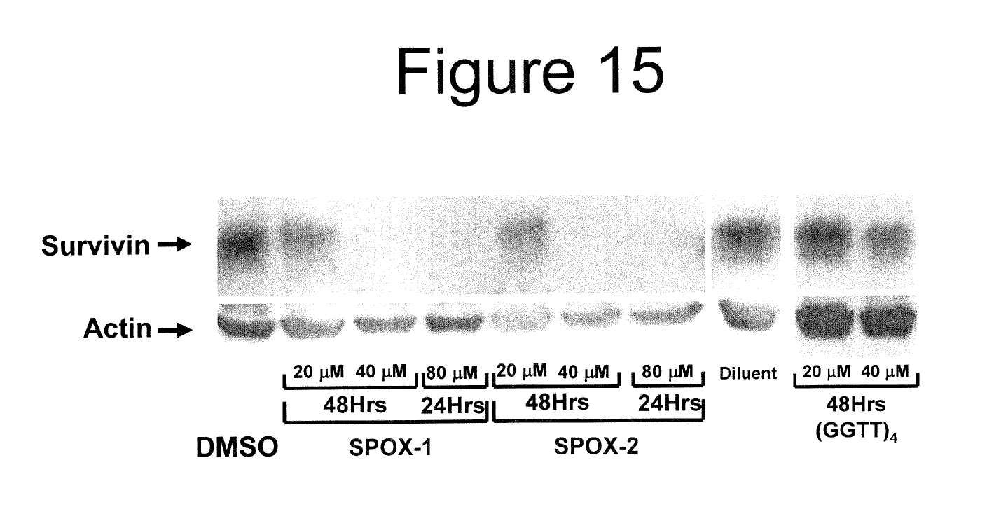

The ability of the SPOX compounds to inhibit expression of survivin, a member of the Inhibitor of Apoptosis Protein (IAP) family, was determined by treating H460 human lung cancer cells for two days with diluent (DMSO) alone, SPOX-1 (20 μM, 40 μM), SPOX-2 (20 μM, 40 μM) and T-oligo (16mer: 5′ GGTTGGTTGGTTGGTT 3′; SEQ ID NO: 3) (20 μM, 40 μM) or for 24 hours with SPOX-1 (80 μM) or SPOX-2 (80 μM). Following treatment, cells were collected, counted, and assayed for survivin expression. The results are depicted in FIG. 15. A dose-dependent reduction in survivin expression was observed at 2 days with both SPOX compounds similar to that observed with 16mer T-oligo, with survivin nearly undetectable at the 40 μM dose. At 24 hours, the 80 μM dose of both SPOX compounds reduced survivin expression to nearly undetectable levels.

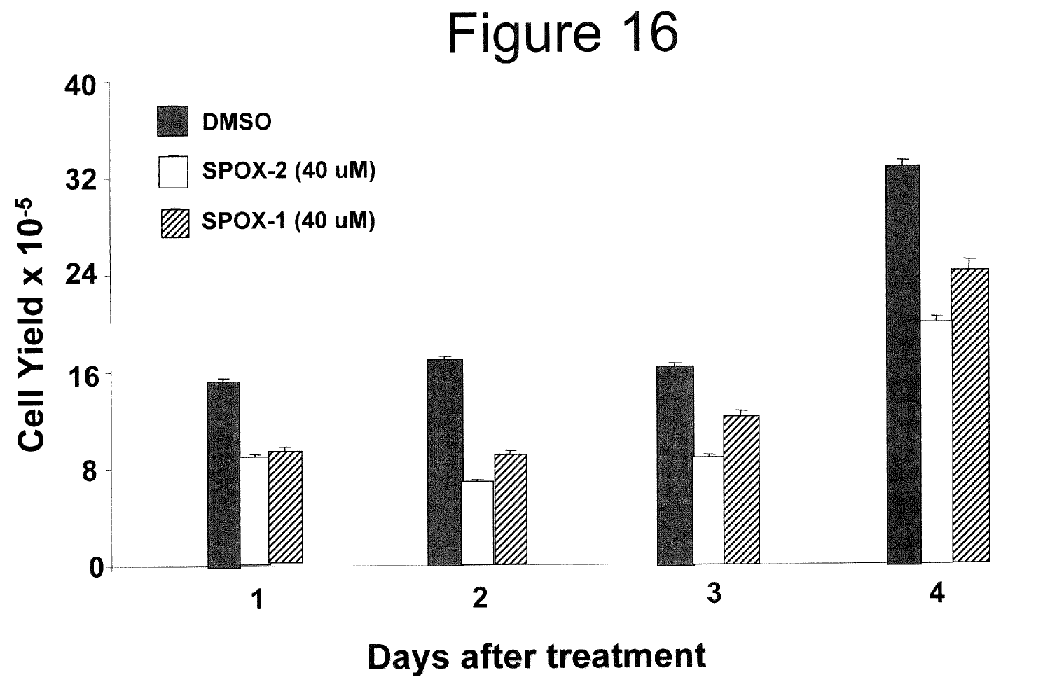

The effect of SPOX compounds on growth of human lung cancer cells was tested using cultures of H460 cells. Briefly, 20,000H460 cells were plated per 35 mm dish. Medium containing either diluent (DMSO) alone, SPOX-1 or SPOX-2 (40 μM) was introduced at 96 hours and growth was determined 1, 2, 3 and 4 days after plating. Both SPOX-1 and SPOX-2 inhibited growth of H460 cells as shown in FIG. 16.

### Example 7

### SPOX-337, SPOX-338 and SPOX-343 Induce Growth Arrest in Human Breast Cancer Cells and Induce Apoptosis in Human Melanoma Cells

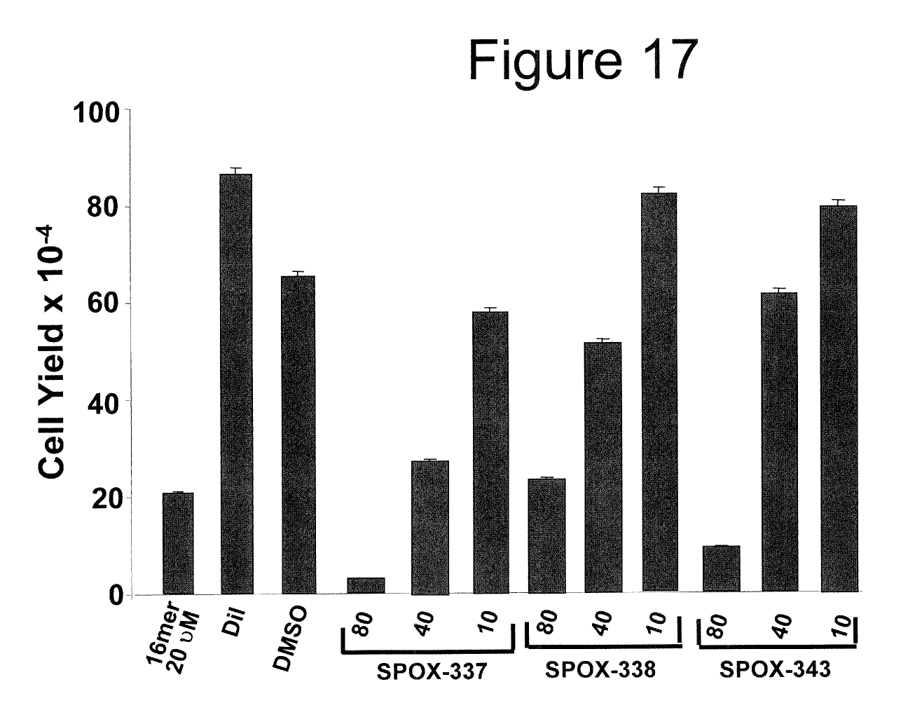

The effect of SPOX compounds on growth of human breast cancer cells was tested using cultures of MCF-7 cells. The effect on cell growth was tested by plating 20,000 cells per 35 mm dish. Medium containing either diluent (water or DMSO) alone, SPOX-337, SPOX-388, SPOX-343 (10 μM, 40 μM and 80 μM) or 16mer T-oligo (SEQ ID NO: 2) was introduced at 96 hours and growth was determined 3 days after plating. SPOX-337, SPOX-338 and SPOX-343 inhibited growth of MCF-7 cells in a dose-dependent manner with the highest concentration of 80 μM leading to nearly complete inhibition of growth as shown in FIG. 17.

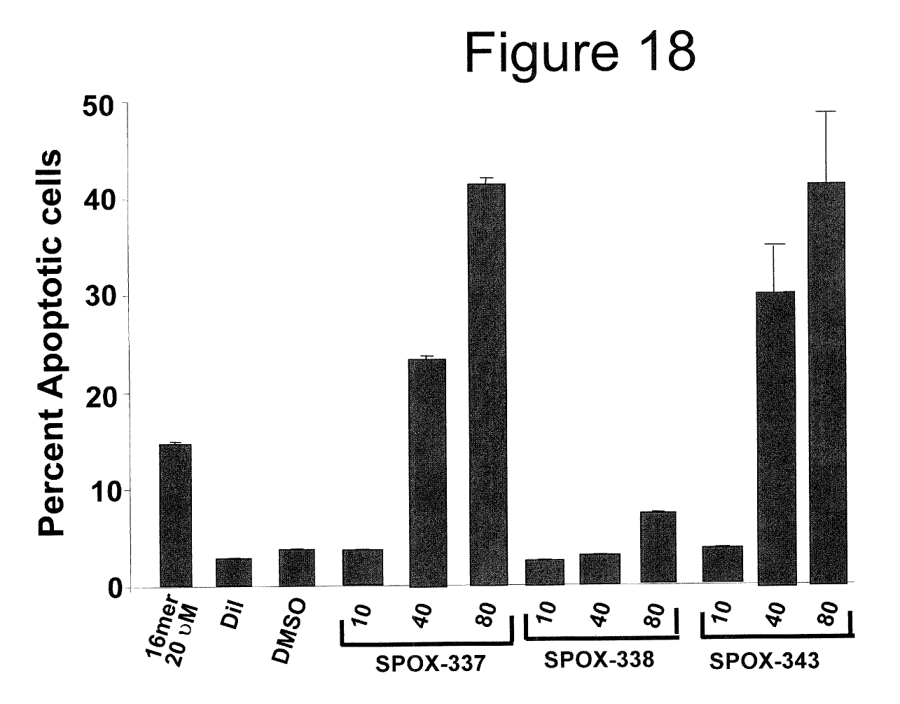

Cultures of MM-AN human melanoma cells were treated with diluent (water or DMSO) alone, 16mer T-oligo (SEQ ID NO: 2), SPOX-337, SPOX-338 or SPOX-343 for 72 hours, then collected and processed for FACS analysis. One concentration of the T-oligo (20 μM) and three concentrations of each SPOX compound (10 μM, 40 μM and 80 μM) were tested. Results are shown in FIG. 18. As can be seen, SPOX-337, SPOX-338 and SPOX-343 induced apoptosis with SPOX-337 and SPOX-343 having greatest effect. The results demonstrate that each compound was able to mimic the effect of 16mer T-oligo on induction of apoptosis in the melanoma cells.

### Example 8

### SPOX-1 and SPOX-2 Induce p53 and p21 in Human Fibroblasts

Human fibroblasts were treated with diluent (water or DMSO) alone, SPOX-1, SPOX-2, or 16mer T-oligo (SEQ ID NO: 2) for 48 hours after which total cellular proteins were harvested and Western blot analysis performed using anti-p53 and anti-p21 antibodies. Compared with diluent alone, SPOX-1 and SPOX-2 induced the level of p53 and induced the level of the p53-dependent downstream effector protein p21 to a similar degree as 16mer T-oligo.

1. A spirooxindole having the general formula: 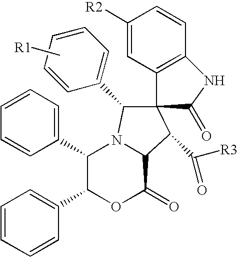
or a pharmaceutically acceptable salt, enantiomer, mixture of enantiomers, or a racemate thereof, wherein:
R1 is in the ortho, meta or para position and is a member selected from the group consisting of: hydroxy, lower alkoxy and hydroxy-substituted lower alkoxy
R2 is a member selected from the group consisting of: hydrogen, lower alkyl halide, halogen, lower alkynyl and substituted lower alkynyl; and
R3 is a member selected from the group consisting of: hydroxy, amino, substituted amino, heterocylic ring, arylheterocyclic ring, lower alkoxy and lower alkenoxy,

2. A spirooxindole in accordance with claim 1 in which:
R1 is in the para position and is hydroxy or hydroxy-substituted lower alkoxy R2 is hydrogen, halogen or substituted lower alkynyl; and
R3 is lower alkenoxy or amino.

3. A spirooxindole in accordance with claim 1 in which:
R1 is in the para position and is hydroxy-substituted lower alkoxy; R2 is hydrogen or halogen; and
R3 is lower alkenoxy.

4. A spirooxindole in accordance with claim 3 in which R3 is —O—CH₂—CH═CH₂.

5. A spirooxindole in accordance with claim 1 in which:
R1 is in the para position and is hydroxy;
R2 is hydrogen; and
R3 is —O—CH₂—CH═CH₂.

6. A spirooxindole in accordance with claim 1 in which:
R1 is in the para position and is —O—CH₂—CH₂—OH₂
R2 is hydrogen; and
R3 is —O—CH₂—CH═CH₂.

7. A spirooxindole in accordance with claim 1 in which:
R1 is in the para position and is —O—CH₂CH₂—OH;
R2 is iodine; and
R3 is —O—CH₂—CH═CH₂.

8. A spirooxindole in accordance with claim 1 wherein the spirooxindole is selected from the group consisting of SPOX-1, SPOX-2 SPOX-343, SPOX-338, SPOX-337 and an enantiomer, mixture of enantiomers, racemate and a pharmaceutically acceptable salt thereof.

9-10. (canceled)

11. A pharmaceutical composition comprising the spirooxindole of claim 1 and at least one pharmaceutically acceptable excipient, diluent, preservative, stabilizer, or mixtures thereof.

12. A method of treating a hyperproliferative disorder in a mammal, the method comprising administering to a mammal in need thereof, an effective amount of a composition comprising a compound selected from the group consisting of a spirooxindole (SPOX) according to claim 1, SPOX-1, SPOX-2, SPOX-343, SPOX-338 and SPOX-337.

13-19. (canceled)

20. The method of claim 12 wherein the composition comprises spirooxindole at concentration of about 1 μM to about 500 μM.

21. A method of inhibiting growth of cancer cells in a human comprising administering to the human a physiologically effective dose of a compound selected from the group consisting of a spirooxindole (SPOX) according to claim 1, SPOX-1, SPOX-2, SPOX-343, SPOX-338 and SPOX-337.

22. The method of claim 21 wherein the cancer cells are selected from melanoma cells, breast cancer cells, lymphoma cells, osteosarcoma cells, leukemia cells, squamous carcinoma cells, cervical cancer cells, ovarian cancer cells, pancreatic cancer cells, and fibrosarcoma cells.

23-28. (canceled)

29. A method of inducing melanogenesis in a mammal, said method comprising administering to the mammal an effective amount of a composition comprising a spirooxindole according to claim 1 and at least one pharmaceutically acceptable excipient.

30. (canceled)

31. A method of inducing apoptosis in cancer cells in a human, said method comprising administering to the human an effective amount of a composition comprising a spirooxindole according to claim 1 and at least one pharmaceutically acceptable excipient.

32. The method of claim 31 wherein the cancer cells are melanoma cells.

33. The method of claim 31 wherein the spirooxindole is selected from the group consisting of the spirooxindole of claim 1, SPOX-1, SPOX-2, SPOX-343, SPOX-338 and SPOX-337.

34-41. (canceled)

42. A method for reducing the occurrence of skin cancer in a human, said method comprising applying to the skin an effective amount of a composition comprising a spirooxindole according to claim 1 and at least one pharmaceutically acceptable excipient.

43. (canceled)

44. A method for reducing the occurrence of skin cancer in a human with xeroderma pigmentosum or other genetic predisposition to skin cancer, said method comprising administering to the skin an effective amount of a composition comprising a spirooxindole according to claim 1.

45-47. (canceled)

48. A method for reducing oxidative damage in a mammal, said method comprising administering to the mammal an effective amount of a composition comprising a spirooxindole according to claim 1 and at least one pharmaceutically acceptable excipient.

49. The method of claim 48 wherein the administration step comprises applying the composition to the skin of said mammal.

50. (canceled)

51. A method for treating melanoma in a mammal, comprising administering to the mammal an effective amount of a composition comprising a spirooxindole according to claim 1 and at least one pharmaceutically acceptable excipient.

52. (canceled)

53. A method for reducing proliferation of keratinocytes in the skin of a human, said method comprising administering to the skin an effective amount of a composition comprising a spirooxindole according to claim 1 and at least one pharmaceutically acceptable excipient.

54. The method of claim 53, wherein the human has actinic keratosis, Bowen's disease, squamous cell carcinoma, or basal cell carcinoma.

55. (canceled)

56. A method of preventing or reducing DNA damage in cells of a mammal, wherein said DNA damage is caused by radiation or DNA-damaging chemicals, comprising contacting said cells with an effective amount of a composition comprising a spirooxindole according to claim 1 and at least one pharmaceutically acceptable excipient.

57. (canceled)

58. A method for inhibiting proliferation of cells in a human comprising administering to the human a physiologically effective dose of a compound selected from the group consisting of a spirooxindole (SPOX) according to claim 1, SPOX-1, SPOX-2, SPOX-343, SPOX-338 and SPOX-337.

59. The method of claim 58, wherein the cells are epithelial cells.
